SEQUENCE LISTING (8 sequences)

SEQ ID NO: 1 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 9 aa, Artificial Sequence
  synthetic
ALENYITAL

SEQ ID NO: 3 - protein, 9 aa, Artificial Sequence
  synthetic
KLVFFAEDV

SEQ ID NO: 4 - protein, 9 aa, Artificial Sequence
  synthetic
LMVGGVVIA

SEQ ID NO: 5 - protein, 9 aa, Artificial Sequence
  synthetic
GLMVGGVVI

SEQ ID NO: 6 - protein, 9 aa, Artificial Sequence
  synthetic
VIVITLVML

SEQ ID NO: 7 - protein, 9 aa, Homo sapiens
RLALENYIT

SEQ ID NO: 8 - protein, 9 aa, Homo sapiens
LALENYITA

(19) United States
(12) Patent Application Publication
(10) Pub. No.: US 2020/0400688 A1
(43) Pub. Date: Dec. 24, 2020

(54) METHODS AND SYSTEMS FOR IDENTIFICATION AND TREATMENT OF PATHOLOGICAL NEURODEGENERATION AND AGE-RELATED COGNITIVE DECLINE

(71) Applicant: CEDARS-SINAI MEDICAL CENTER, Los Angeles, CA (US)

(72) Inventor: CHRISTOPHER WHEELER, Los Angeles, CA (US)

(73) Assignee: CEDARS-SINAI MEDICAL CENTER, Los Angeles, CA (US)

(21) Appl. No.: 16/968,795

(22) PCT Filed: Feb. 13, 2019
(86) PCT No.: PCT/US19/17879

§ 371 Date: Aug. 10, 2020

Related U.S. Application Data
(60) Provisional application No. 62/630,129, filed on Feb. 13, 2018.

(51) Int. Cl. G01N 33/68; A61K 39/00; C07K 14/705; C07K 14/57

(52) U.S. Cl. CPC G01N 33/6896; A61K 39/001128; C07K 14/70546; G01N 2333/70596; C07K 14/57; G01N 2333/70517; A61K 39/0007

(57) ABSTRACT

Methods and systems for diagnosing, providing prophylaxis, and treating one or more age-related neurodegeneration or pathological cognitive impairment are provided. An increased presence of CD103+ resident memory CD8+ T cells (CD8+ T_{RM}) can be detected in blood sample obtained from the human subjects with one or more symptoms of loss of short-term or long-term memory, decreased ability to maintain focus, and decreased problem-solving capacity. An increased presence of CD103+ CD8+ T_{RM} can be compared to a value obtained from one or a pool of healthy human subjects with none of the one or more symptoms. One or more of a therapeutically effective amount of: an inhibitor of cluster of differentiation (CD103), an inhibitor of perforin-1, and an inhibitor of interferon gamma (IFNγ) can be administered as treatment of age-related neurodegeneration or pathological cognitive impairment. Pathological neurodegeneration can include Parkinson's disease, multiple sclerosis, or Alzheimer' s disease.

### CROSS-REFERENCE TO RELATED APPLICATIONS

This application claims priority under 35 U.S.C. 371 to International Patent Application PCT/US2019/017879 filed Feb. 13, 2019 and further claims priority under 35 U.S.C. § 119(e) to U.S. provisional patent application No. 62/630,129, filed on Feb. 13, 2018, the content of which are herein incorporated by reference in its entirety.

### FIELD OF INVENTION

This invention relates to diagnostic, preventative care and treatment of age-related or pathological neurodegeneration.

### BACKGROUND

Aging contributes to the onset and/or progression of multiple diseases, but the specific aspects of aging affecting individual disease dynamics remain largely mysterious. Chronic inflammation is increasingly recognized as a critical contributor to a variety of age-related diseases, including cancer, cardiovascular disease, cancer, and neurological conditions such as stroke, trauma and neurodegeneration.

T cells are master regulators of inflammation throughout the body, and their mis-regulation promotes chronic inflammation. CD8+ T cells can acquire self-destructive potential, especially as they undergo homeostatic expansion when the peripheral T cell pool is depleted. Such depletion occurs gradually during aging as the production of new T cells by the thymus wanes, or more acutely due to stress, trauma or infection. Age-related T cell expansions is continual and almost ubiquitous by late middle age in humans, making it difficult to study the pathological involvement in aberrant T cell expansion in lieu of the natural aging process. Moreover, aberrant age-related T cell expansion is relatively infrequent in experimental rodents, and its functional impact, if any, is often offset by the persistent thymic activity in aging rodents.

Introducing T cells into lymphopenic hosts results in their immediate homeostatic expansion, and the resulting cells can exacerbate induced autoimmunity in experimental rodents. Nevertheless, the relationship is poorly defined between this more rapid expansion and the age-related T cell aberrancies, and in most cases is not known to promote recognizable age-related disease.

Aberrant CD8+ T cell clones in particular expand in most aging humans, whereas this process is offset by compensatory processes in aging mice. Changes in memory CD8+ T cells are also among the explicit physiological differences observed between mouse models, in which these cells are decreased, and human Alzheimer's disease, where they are increased. Moreover, recent studies convincingly demonstrate that memory CD8+ T cells increase in the circulation and/or central nervous system (CNS) of Alzheimer's disease patients. These increases commonly accompany prominent changes in other T cells, but they tend to correlate better with tauopathy and/or cognitive decline. Thus, age-related homeostatic expansion, and the abnormal memory CD8+ T cells it produces, may represent a missing physiological factor in mice that could impact resistance to full Alzheimer's disease pathology. Testing this remains challenging, however, because of the pervasiveness of aberrant aged CD8+ T cells in healthy humans, their scarcity in mice, and their co-occurrence with other aging features in all species.

Therefore, it is an objective of the present invention to provide a composition and a process of using thereof for the diagnosis, prophylaxis, and/or treatment of age-related cognitive decline including pathological neurodegeneration.

It is also an objective of the present invention to provide a composition and a process of using thereof for screening candidate therapeutic, prophylactic and/or diagnostic agents for human cognitive decline in an in vitro system or in a rodent model.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

A process is provided including administering an inhibitor of CD103, an inhibitor of the effector molecules of CD8+ T_{RM} (CD8+ resident memory T cells), and/or a tolerogenic vaccine to protect against and/or reduce the severity of cognitive decline in an at-risk elderly subject, a mild cognitive impaired subject, and/or a subject with neurodegenerative disease. In various embodiments, the CD8+ T_{RM} can be reactive to, or specific to, amyloid precursor protein (APP) or an APP peptide. An aspect of the process provides administering an inhibitor of CD103, an inhibitor of perforin-1, an inhibitor of interferon γ (IFNγ), and/or a vaccine including APP peptides can reduce the binding or reaction of CD8+ T_{RM} to APP or APP peptides, compared to prior to the administration of the one or more inhibitors or vaccine, or compared to a control subject not receiving an administration of the one or more inhibitors

The inhibitors include an antibody or a fragment thereof, a small molecule, or a nucleic acid. In some embodiments, the inhibitor can be an anti-CD103 antibody, such as 2G5.1, a mouse anti-human IgG2a monoclonal antibody, or a humanized antibody of 2G5.1. In other embodiments, the inhibitor can inhibit perforin-1 or inhibit interferon-gamma. In some embodiments, the tolerogenic vaccine can include an amyloid precursor protein or an peptide thereof.

A process is provided for identifying subjects susceptible to or experiencing age-related neurodegeneration, including Alzheimer's disease, where the process can include detecting increased levels of CD103-positive resident memory T cells (T_{RM}) in blood.

A kit is provided including a sample collection device and optionally a manual of operation to collect and quantify CD103-positive CD8+ T_{RM} for use by a subject prior to, during and/or in-between treatments of memory disorders.

A system is provided to identify and/or screen candidate therapeutic, prophylactic and/or diagnostic agents for human cognitive decline or age-related neurodegeneration, where the system can include CD44^{hi}CD123⁺CD127^{hi}KLRG1⁺CD103⁺ resident memory CD8+ T cell phenotype that is obtained from a rodent (e.g., mouse) animal. In some embodiments, the CD44^{hi}CD123⁺CD127^{hi}KLRG1⁺CD103⁺ resident memory CD8+ T cell phenotype can be obtained by administering resident memory CD8+ T cells in a thymus-deficient mouse.

A process of identifying and/or screening a candidate agent for human therapeutics or prophylaxis of age-related neurodegeneration can include contacting the candidate agent with the CD44^{hi}CD123⁺CD127^{hi}KLRG1⁺CD103⁺ resident memory CD8+ T cells in vitro or administering the candidate agent to a model animal containing the CD44^{hi}CD123⁺CD127^{hi}KLRG1⁺CD103⁺ resident memory CD8+ T cells, to identify a reduced level of CD103-positive resident memory CD8+ T cells, a reduced level of their effector molecules, or a reduced amount of the emigration of CD8+ T cell from the periphery system to the brain of the animal.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1G depict that ^{hi}T cells exhibit an age-related, resident memory phenotype (^{hi}T_{RM}). FIGS. 1A-1D are on spleen specimen, whereas FIGS. 1E-1G are on brain analysis. Representative flow cytometry analysis of age-related markers on splenic CD8+ T cells from young (<10 weeks) and old (>12 months) C57BL/6 (B6), (denoted “Young B6” and “Old B6”, respectively), and young (6 weeks) B6.Foxn1 recipients of i.v. CD8+ T cells (denoted “CD84→B6.Foxn1”) 3-5 weeks after injection (FIG. 1A). Percentage of lymphocytes (FIG. 1B) and mean fluorescence intensity (FIGS. 1C and 1D) from flow cytometry compiled from n≥6 mice/group. Proportions of mice with “diverse” TCRVβ D→J gene segment usage (>3 segments/brain) and specific D→J segments within brains of young (<10 weeks, denoted “young B6”), middle-aged (6 months, denoted “middle-age B6”), and old (>12 months, denoted “old B6”) B6 mice, reveals an age-dependent pattern of progressively decreased diversity and increased usage of particular D→J segments (i.e., clonality; FIGS. 1E and 1F; columns appear in the order defined from left to right below the respective figure). D→J diversity and segment usage was significantly correlated only between old B6 and young CD8→B6.Foxn1 brain (FIG. 1G). *P<0.05, **P<0.01, ***P<0.005 by 2-sided T-test n≥5 mice/group in ≥3 independent tests for flow cytometric markers, and by Pearson's correlations in n≥10 mice/group for PCR compilations.

FIGS. 1H-1K depict the expansion of donor cells in B6.Foxn1 mice deficient for amyloid precursor protein (APP). Purified CD8+ T cells from female C57BL/6 or congenic knockout hosts were injected into 8-10 week-old female B6.Foxn1, B6.Foxn1-AppKO, or B6.CD45.1-congenic recipients (FIG. 1H). Blood was analyzed by flow cytometry 3 days later using the gating and antibodies to T cell markers as shown (FIG. 1I), with % CD3ε⁺CD8+ in gated cells compiled in (FIG. 1J). B6.Foxn1 mice were crossed to B6.App-knockout mice, homozygous double-mutants (B6.Foxn1-AppKO) verified by PCR and phenotype at Jackson Laboratories (Bar Harbor, Minn.), and CD8+ T cells expansion assessed by CFSE dilution in B6.Foxn1 and B6.Foxn1-AppKO female recipients (FIG. 1K; n=3 B6.Foxn1 & n=5 B6.Foxn1-AppKO; *P<0.04, ***P<0.00001 by 2-tailed T-test in 3 independent trials; n≥5 mice/group in ≥3 independent tests for all markers).

FIGS. 2A-2E depict that ^{hi}T_{RM} are reactive to self-antigens, and selectively enter brain (i.e., brain CD8+ T cell phenotype after transfer into nude mice). Light scatter and gating of brain lymphocytes and CD8+ T cells in B6.Foxn1 recipients (FIG. 2A). Percentage and phenotype of CF SE+CD8+ T cells within brain lymphocytes in B6.Foxn1 recipients 3 days (FIG. 2B), and 10 weeks (FIG. 2C) after injection. Increased staining with pMHC I multimers (custom dextramers synthesized by Immudex USA, Fairfax, Va.) to Trp-2-DCT₍₁₈₀₋₁₈₈₎/H-2K^{b} and APP₍₄₇₀₋₄₇₈₎/H-2D^{b} epitopes on KLRG1⁺ CD8+ T cells in B6.Foxn1 brain (FIGS. 2D and 2E) and spleen (FIG. 2E), 10 weeks after injection (*P<0.05 by 2-sided T-test in ≥3 independent tests; n>6 for all analyses, with significance relative to PBS group).

FIGS. 2F-2J depict that ^{hi}T_{RM} induction increases CD 8 and amyloid precursor protein (APP)/Ab in brain (i.e., PCR and Western analysis of T cell and amyloid markers). PCR for TCRVβ D1→J1 (FIG. 2F) and D2→J2 (FIG. 2G) gene segments indicated diverse T cell repertoires in young and old C57BL/6 (B6), but restricted TCRVβ diversity in B6.Foxn1 recipients of CD8+ T cells after 10 weeks (CD8→B6.Foxn1). B6.Foxn1 mice lacked rearranged (i.e., visibly contained only germline; “G”) TCR products in brain unless wt-CD8+ T cells were injected previously (FIGS. 2F and 2G; note, segment J2.6 is a pseudogene). Western blot of CD8α (antibody clone 2.43; FIG. 2H) and beta amyloid (antibody clone 4G8; FIG. 2I) in dissected brain hippocampus of young (<5 months) C57BL6 (B6) and B6.Foxn1 hosts with and without adoptive transfer of CD8+ T cells from young (6-8 wk) B6 donors 10 weeks prior. CD8 protein is detectable at very low levels in B6, but is undetectable in B6.Foxn1 unless wt-CD8= T cells were injected 10 weeks earlier. “Ref”=6-10 week-old female C57BL/6 spleen DNA or cell lysate, subjected to identical analysis. FIG. 2J depicts the timeline of the study.

FIGS. 3A-3J depict Aβ plaque and neurofibrillar pathology in nude mice harboring hiT cells. Westerns of detergent-soluble APP cleavage products (APP^{Cl}) in dissected cortex and hippocampus 3 wk after control or cell injection (→) in indicated recipients (FIG. 3A). Cell/control recipients in FIGS. 3B-3J are B6.Foxn1 exclusively, with time after injection at 15 mos unless otherwise indicated. Forebrain ELISA of Triton-soluble Aβ1-40/42 (FIG. 3B). Parenchymal plaques with and without pTau or curcumin counter-staining (FIG. 3C), and compiled 4G8 burden (FIG. 3D) in entorhinal (Ent) and cingulate (Cng) cortex, and hippocampus (Hippo) in indicated mouse groups. Forebrain Westerns (FIG. 3E), and compiled signal quantification (FIG. 3F), of detergent-soluble phospho-tau (pTau) and paired helical filaments (PHF). Silver-stained cells in brain, and in 18 month-old ADtg (Tg2576) mice, with sequential pTau→Gallyas stains inset (FIG. 3G). Compiled proportions of Gallyas⁺ neurons (FIG. 3H), astrocytes (Gfap⁺), and microglia (Iba-1⁺; FIGS. 3I and 3J). *P<0.05, **P<0.01, ***P<0.005 by 2-sided T-test in ≥3 independent tests for all analyses, relative to PBS group.

FIGS. 3K and 3L depict that ^{hi}T_{RM} induce fibrillar inclusions in brain cells at 6 months (i.e., distinct curcumin and ThioS staining in dentate gyms of nude mice harboring hiT cells). Hippocampal sections from the indicated groups (all B6.Foxn1 recipients, except AD-Tg=Tg2576 mice), were stained for 4G8 (Aβ) and curcumin, 6 months after i.v. control/cell injection, or at 14 months of age for AD-Tg (FIG. 3K). Right-facing arrows highlight Aβ deposits with no curcumin co-staining. Up-facing arrows depict co-localized Aβ and curcumin deposits, representing mature neuritic plaques. Down-facing arrows highlight curcumin⁺ structures with no Aβ co-staining, i.e., non-amyloid fibrillar deposits. No DAPI was used in the stains; blue channel background is provided for anatomical context only. Follow-up ThioS staining of PBS and wt-CD8 group B6.Foxn1 hiT recipients 6 months after control/cell injection, and 20 month-old AD-Transgenic (Tg) rat dentate gyms (FIG. 3L). Rat AD-Tg brain was used due to its explicit Tau PHF content (Cohen et al., 2013), that nevertheless failed to stain with ThioS in our hands.

FIGS. 3M and 3N depict the silver stained neuronal structures in experimental groups. Gallyas silver staining of cortical and hippocampal brain regions, showing typical neurofibrillary tangle (NFT) morphology in wt-CD8 and IFNγKO-CD8 group mice (insets). Background silver staining was occasionally evident in PrfKO-CD8 or PBS group mice, but did not exhibit similar NFT morphology (insets). Individual images were derived from different mice within each group (FIG. 3M). Comparison of Gallyas⁺structures in nude mice harboring hiT cells (wt-CD8) hippocampus (left) and cortex (ctx, right), to those in cortex of human severe AD (Braak stage VI; FIG. 3N). Magnification and scale are identical for all images (20×), and among insets in FIGS. 3M and 3N.

FIGS. 3O-3S depict the brain CD8+ T cells in ^{hi}T_{RM}-recipient B6.Foxn1 mice. Brain was co-stained for CD8 and pTau (inset)(FIG. 3O), and quantified within hippocampal and cortical brain sections from B6.Foxn1 recipients 15 months after injection of wild-type, IFNγKO or PrfKO CD8+ T cells, or PBS. CD8⁺ cells, though mostly solitary, were occasionally seen interacting with pTau⁺ neurons as in FIG. 3O (inset). Group data are compiled in FIG. 3P. Astrocytic (GFAP, FIG. 3Q), microglial (Iba-1, FIG. 3R), or CD8+ T cell (CD8, FIG. 3S) areas significantly altered in FIGS. 3A-3J or FIGS. 3O-3S (**P<0.01, *P<0.05; 2-sided T-test relative to PBS control) were correlated with 4G8⁺ plaque burden within each group, with P values of linear regressions and Pearson's correlations (r) shown (FIGS. 3Q-3S).

FIGS. 4A-4N depict the neurodegenerative metrics and cognition in nude mice harboring hiT cells (i.e, in ^{hi}T_{RM}-recipient B6.Foxn1 mice). Cell/control recipients in all panels are B6.Foxn1 exclusively. NeuN and GFAP staining (FIGS. 4A and 4B), and cell counts in hippocampus, 15 mos after cell/control injection (FIG. 4C). Brain atrophy over time in PBS and wt-CD8 groups (mass normalized to PBS controls at each time point; FIG. 4D). Representative forebrain Westerns (FIG. 4E), and GAPDH-normalized NeuN, Drebrin, and Synaptophysin Western signals (FIG. 4F). Correlation of NeuN with brain weight (FIG. 4G). Representative Open Field test at 13 months (FIG. 4H). Fear Conditioning performance over time (FIG. 4I), and Spontaneous Alternation (SA) at 12 months (FIG. 4J). Barnes Maze learning (FIG. 4K; P from 2-sided ANOVA), retention (FIG. 4L), and reversal (FIGS. 4M and 4N) phases, at 14 mos (black, colored symbols=P relative to PBS, wt-CD8, respectively). ***P<0.005, **P<0.01, *P<0.05, ⁺P<0.1 by 2-tailed T-test, except where other tests are indicated.

FIGS. 5A-5D depict CD103-deficiency primarily impacts CD8+ T cells and brain localization. Flow cytometry (FIG. 5A), Western blot (FIG. 5B), Western signal compilation (FIG. 5C), and Open Field Test results (FIG. 5D; columns appear in the order defined from top to bottom on the right hand side of this figure), from CD103-deficient (B6.CD103KO) and age-matched wild-type (B6) mice (n=8). CD103-deficiency primarily impacted CD8+ T cells (FIG. 5A), while specifically decreasing CD8 signal within brain (FIGS. 5B and 5C; CD8+ T_{RM} otherwise specifically increase in aging brain), and slightly slowing locomotion with aging (FIG. 5D).

FIGS. 5E-5H depict CD103-deficiency protects against age-related cognitive decline. Performance of young and old CD103-deficient and wild-type mice in the Barnes Maze: training phase Latency (FIG. 5E); memory retention phase Latency (FIG. 5F); reversal phase Latency (FIG. 5G); and Entry Errors at each phase (FIG. 5H). The major age-related defect in this strain on this test has been reported to be entry errors.

FIGS. 6A-6F depict the increased hiT cell-associated metrics in human Alzheimer's brain. FIG. 6A depicts the GFAP expression units in the brain of subjects with no Alzheimer's disease (no AD) compared to in the brain of subject with AD. FIG. 6B depicts the change (%) of gene expression levels, relative to the corresponding GFAP expression level in the subjects, in the brain. PRF1 Western and immunofluorescence (FIG. 6C), with quantifications in age-matched normal (n=6), mild (n=5), or severe (n=12) Alzheimer's disease brain (FIG. 6D). Alzheimer's disease brain co-stained with anti-CD8 (Serotec) and APP₍₄₇₁₋₄₇₉₎/HLA-A2 multimer (Immudex USA, Fairfax, Va.)(FIG. 6E), with quantification of epitope-reactive T cells (P=0.002, 2-sided T-test)(FIG. 6F). Overall levels of CD8+ T cells were unchanged (1.63±0.29 vs. 2.29±0.55 cells/vessel in Alzheimer's disease vs. normal aging controls; P=0.31, 2-sided T-test).

FIGS. 7A and 7B depict the levels of aberrant, APP-specific CD8+ T_{RM} in patients at risk of (MoCA=26-30) and experiencing (MoCA<26) age- or pathology related cognitive decline, within all patients (FIG. 7A), and within HLA-A2⁺ (potentially APP epitope-reactive) specifically (FIG. 7B).

FIG. 8 depicts a general model for CD8+ T_{RM}-mediated brain effects.

FIGS. 9A-9C depict CD8+ Tim gene expression in human patient blood (FIGS. 9A and 9B) and ^{hi}T_{RM} staining metrics in human patient blood (by flow cytometry) (in FIG. 9C, columns appear in the order defined from top to bottom at the top right corner of the figure).

FIGS. 10A-10E depict the expression of aberrant aged T cell genes in normal aging human subject's blood and in Alzheimer's human patients' blood (gene Expression Omnibus dataset GSE85426). FIGS. 10A-10C depict three key genes CD103, CD8A, CD44, respectively, are significantly increased in Alzheimer's Disease. Patients with lower-than-average expression of a general T cell gene CD3D (FIG. 10D) were excluded from biomarker analysis to ensure predictive power is T cell-dependent. Patients younger than 65 years old (FIG. 10E) were also excluded to eliminate rare early-onset forms of Alzheimer's Disease (AD) with distinct genetic causes. P values less than 0.05 (*) represent statistically significant differences between normal and AD patients; **** represents P<0.0005.

FIGS. 11A and 11B depict true vs. false-positive prediction rates for Alzheimer's Disease (AD) by the 3-gene panel (CD8, CD44, CD103). After exclusions (described in the previous paragraph), there were 40 normal and 49 AD specimens left for biomarker analysis in the T cell-high group (FIG. 11A), and 40 normal and 39 AD specimens in the T cell-low group (FIG. 11B). The accurate prediction of at least 40% of AD patients with <5% false-positive predictions in T cell-high specimens validates that aberrant aged T cells are relevant to AD, and serve as a potent blood biomarker for late-onset forms of the disease. Including CD8A and CD44 in the receiver operating characteristic (ROC) analysis with CD103 more specifically identifies the CD8+ T_{RM} subpopulation than just CD103 alone. Compared with the respective ROC curve based on CD103 alone shown in FIGS. 12A and 12B, the respective ROC curve in FIG. 11A or 11B is not substantially (or significantly) different, indicating that CD103 is also highly specific for this subpopulation, while the slightly lower false-positive rate for the 3-gene panel indicates that further increasing specificity for T_{RM} identification will further improve its biomarker value.

FIGS. 12A and 12B depict true vs. false-positive prediction rates for Alzheimer's Disease (AD) by CD103 alone from specimens with the same exclusions as described in relation to FIGS. 11D and 11E for plotting of the T cell-high group (FIG. 12A) and the T cell-low group (FIG. 12B). Accurate prediction of over 50% of AD patients with <8% false-positive predictions in T cell-high specimens validates that CD103 on T cells is relevant to AD, and serves as a single-gene blood biomarker for late-onset forms of the disease.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

T cells can be distinguished from other lymphocyte types, such as B cells, by the presence of a special receptor on their cell surface called a T cell receptor (TCR). Several different subsets of T cells have been discovered, each with a distinct function including T helper cell (T_{H} cells), cytotoxic T cells (Tc cells, or CTLs), memory T cells (T_{M} cells) including central memory T cells (T_{CM} cells) and effector memory T cells (T_{EM} cells), natural killer T cells (NKT cells), gamma delta T cells (γδT cells), and regulatory T cells (T_{reg} cells).

CD8+ T cells express the CD8 glycoprotein at their surface. Most T_{C} cells (also known as CD8+ T cells) express TCRs that recognize a specific antigen. An antigen (usually a peptide resulting from intracellular degradation of a protein) inside a cell forms a complex with a Class I WIC molecule, and is then brought to the surface of the cell with the Class I MHC molecule, where they can be recognized by a T_{C} cell. If the T_{C} cell's TCR is specific for that antigen, the T_{C} cell binds to the complex of the MHC molecule and the peptide, and the T_{C} cell destroys the cell. The affinity between CD8 and the WIC molecule keeps the T_{C} cell and the target cell bound closely together during this antigen-specific activation. CD8+ T cells are recognized as T_{C} cells once they become activated and are generally classified as having a pre-defined cytotoxic role within the immune system.

“APP” as used herein refers to amyloid precursor protein. “APP peptide” as used herein refers to a peptide comprising a portion of APP amino acid sequence. The peptides may be 2 to 20 amino acids long (e.g., 2, 3, 4, 5, 6, 7, 8, 9, 10, 11, 12, 13, 14, 15, 16, 17, 18, 19, or 20 amino acids long). In further embodiments, APP peptide suitable for use with aspects of the invention described herein may be derived from the human APP having the full length sequence set forth in SEQ ID NO: 1. In an exemplary embodiment, an APP peptide comprises the sequence ALENYITAL (SEQ ID NO: 2), KLVFFAEDV (SEQ ID NO: 3), LMVGGVVIA (SEQ ID NO: 4), GLMVGGVVI (SEQ ID NO: 5), VIVITLVML (SEQ ID NO: 6), RLALENYIT (SEQ ID NO: 7; amino acid 470-478 of APP) or LALENYITA (SEQ ID NO: 8; amino acid 471-479 of APP). Application publication No. WO/2017/040594 provides further description of APP or APP peptide, the content of which application publication is incorporated by reference herein. In some embodiments, SEQ ID NOs: 2-6 represent APP-derived peptides that may stably bind the most common HLA allele in the western world (HLA-A2) that may be readily manufactured; additional peptide/HLA combinations may be utilized depending on patient cohort demographics as would be apparent to a person of skill in the art.

“Amino acid” as used herein is meant to include both natural and synthetic amino acids, and both D and L amino acids. “Standard amino acid” means any of the twenty L-amino acids commonly found in naturally occurring peptides. “Nonstandard amino acid” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides disclosed herein, and particularly at the carboxy- or amino-terminus, can be modified by methylation, amidation, acetylation or substitution with other chemical groups which can change the peptide's circulating half-life without adversely affecting their biological activity. Additionally, a disulfide linkage may be present or absent in the peptides disclosed herein.

Herein, “peptide” and “protein” are used interchangeably, and refer to a compound comprised of at least two amino acid residues covalently linked by peptide bonds or modified peptide bonds (e.g., peptide isosteres). No limitation is placed on the maximum number of amino acids which may comprise a protein or peptide. The amino acids comprising the peptides or proteins described herein and in the appended claims are understood to be either D or L amino acids with L amino acids being preferred. The amino acid comprising the peptides or proteins described herein may also be modified either by natural processes, such as posttranslational processing, or by chemical modification techniques which are well known in the art. Modifications can occur anywhere in a peptide, including the peptide backbone, the amino acid side-chains and the amino or carboxyl termini. It is understood that the same type of modification may be present in the same or varying degrees at several sites in a given peptide. Also, a given peptide may contain many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold F, Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors,” Meth. Enzymol. (1990) 182: 626-646 and Rattan et al. (1992), “Protein Synthesis: Posttranslational Modifications and Aging,” Ann NY Acad Sci 663: 48-62.

“Sample” or “biological sample” as used herein refers to tissues or body fluids removed from a mammal, preferably human, and which contain or are believed to contain CD8³⁰ T cells. Samples can be blood and/or blood fractions, including peripheral blood sample like peripheral blood mononuclear cell (PBMC) sample or blood (e.g., whole blood, plasma, serum), bone marrow cell sample, or cerebral spinal fluid (CSF). Samples can also be a biopsy of brain tissue. A sample can also include any specific tissues/organ sample of interest, including, without limitation, lymphoid, thymus, pancreas, eye, heart, liver, nerves, intestine, skin, muscle, cartilage, ligament, synovial fluid, and/or joints. The samples can be taken from any individual including a healthy individual or an individual having cells, tissues, and/or an organ afflicted with the unwanted immune response. Methods for obtaining such samples are well known to a person of ordinary skill in the art of immunology and medicine. They include drawing and processing blood and blood components using routine procedures, or obtaining biopsies from the bone marrow or other tissue or organ using standard medical techniques.

T cells are master regulators of inflammation throughout the body. T cell mis-regulation promotes chronic inflammation, which is increasingly recognized as a critical contributor to a variety of human diseases. The CD8 memory subsets expand aberrantly with aging and increase in some tissues including brain. However, their expansion occurs infrequently in aging experimental animals, and the functional consequences are also offset by the persistent thymic activity.

Homeostatic expansion generally relies on the recognition of self-antigens and/or cytokines, and as such can promote autoimmunity. It is conceived that aberrant autoreactive CD8⁺ T cells contribute to the initiation or progression of discrete age-related inflammatory pathologies.

Herein to get around limitations of common experimental rodent models, homeostatic expansion in CD8+ T cells is induced by injection into thymus-deficient mice to acquire an age-associated, CD44^{hi}CD123⁺CD127^{hi}KLRG1⁺CD103⁺ resident memory phenotype. The resulting homeostatically-induced (hiT) cells exhibit not only the signature age-related surface marker changes and TCR Vβ clonality, but also reactivity to central nervous system's self-antigens including amyloid precursor protein (APP) and Dopachrome Tautamerase/Trp-2, thereby making feasible the study of neuropathology in hiT-cell recipients. As with normal CD8+ T_{RM} cells, these homeostatically-induced resident memory (^{hi}T_{RM}) cells populated brain, where they surprisingly conferred progressive neuropathology, including (i) increased APP cleavage products and (ii) diffuse beta-amyloid (Aβ) plaques in brain, (iii) fibrillary inclusions in neurons, (iv) neuro-inflammation, and (v) cognitive impairment with age, while losing neurons, synaptic markers and brain mass. Early ^{hi}T exhibits a pro-inflammatory function that leads to neurodegeneration and cognitive impairment in nude mice. Reduction of CD8+ T cells in brain via CD103 deficiency inhibits age-related cognitive decline in immune-sufficient mice. Moreover, ^{hi}T_{RM} epitope specificity was altered both in Alzheimer's brain, and in blood of cognitively impaired patients, which indicates its involvement in pathological and age-associated cognitive decline in humans.

### Composition

In various embodiments, the present invention provides a composition for the prophylaxis or therapeutic treatment of age-related neurodegeneration including pathological neurodegeneration. The composition includes an inhibitor of CD103, an inhibitor of the effector molecules of CD8+ T_{RM}, and/or a tolerogenic vaccine. “Prophylaxis” as used herein include, but is not limited to, reducing the likelihood of having or delaying the onset of the disease or condition.

CD103, also known as integrin alpha E, is an integrin protein that in human is encoded by the ITGAE gene and is the α subunit of the integrain α_{E}β₇ (also known as CD103). CD103 defines a subtype of memory CD8⁺ T cells, called tissue resident memory T (T_{RM}) cells, that stably reside in tissues such as peripheral non-lymphoid tissues, including lung, gut, and skin, where they orchestrate a highly protective local immune response to persistent viral infections.

In some embodiments, the inhibitor of CD103 is an anti-CD103 antibody or an antigen-binding fragment thereof. Exemplary anti-CD103 antibodies for prophylaxis or therapeutic treatment of neurodegeneration include (1) PE anti-human CD103 antibody from clone Ber-ACTB (BIOLEGEND®); (2) a mouse anti-human CD103 monoclonal antibody (mAb) from clone 2G5.1 (BIORAD®); or a humanized antibody of 2G5.1; (3) OX-62, an anti-rat CD103 mAb; or a humanized antibody of OX-62; and (4) an anti-mouse CD103 mAb from clone 2E7 (EBIOSCIENCE™); or a humanized antibody of 2E7.

In other embodiments, the inhibitor of CD103 is a small molecule blocking the activity of CD103 on CD8+ T_{RM}. In yet another embodiment, the inhibitor of CD103 is a nucleic acid that silences or cleaves the DNA or the mRNA corresponding to CD103. In another embodiment, the inhibitor of CD103 is paxillin, a protein which at least binds to the cytoplasmic domain of CD103.

In some embodiments, an inhibitor of the effector molecules of CD8+ T_{RM} is administered for treating, inhibiting, reducing the severity of or promoting prophylaxis of age-related cognitive decline, pathological neurodegeneration, or both, and the inhibitor of the effector molecules is a small molecule, an antibody or a fragment thereof, or a nucleic acid, which blocks the activity or ablates the expression level of perforin-1, interferon-gamma or other inflammatory cytokines released by APP-specific CD8+ T_{RM}-converted effector T cells. One aspect provides an inhibitor of perforin-1 is administered, and the inhibitor of perforin-1 includes but is not limited to diarylthiophenes and GSK2126458. Another aspect provides an inhibitor of interferon gamma (IFNγ) is administered, and the inhibitor of IFNγ includes but is not limited to mesopram and rocaglamide.

In yet other embodiments, the tolerogenic vaccine includes a vaccine delivering an effective amount of amyloid precursor protein or a peptide thereof, and the peptide includes but is not limited to those of SEQ ID Nos: 2-8.

### Pharmaceutical Composition

In various embodiments, the present invention provides a pharmaceutical composition for prophylaxis or treatment of age-related neurodegeneration. The pharmaceutical composition includes an inhibitor of CD103, an inhibitor of the effector molecules of CD8+ T_{RM} such as an inhibitor of perforin-1 and an inhibitor of IFNγ, and/or a tolerogenic vaccine; and a pharmaceutically acceptable excipient. In an embodiment, the inhibitor of CD103 is an anti-CD103 antibody. In another embodiment, the effector molecules include perforin, interferon-gamma or other inflammatory cytokines. In yet another embodiment, the tolerogenic vaccine includes APP or an APP peptide such as those of SEQ ID Nos: 2-8.

The pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

The pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl di stearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

### Kit

In one embodiment, a kit comprises components necessary for identifying, isolating, and/or enriching a population of CD103-positive CD8+ T_{RM} from a biological sample. In another aspect of the embodiment, a kit may further include positive and/or negative controls and/or instructions for identifying, isolating, and/or enriching of CD103-positive CD8+ T cells by use of the kit's contents. In still other aspects of this embodiment, the kit may further include culture containers like dishes or flasks, culture medium, or any necessary buffers, factors, useful to promote cell growth.

In another embodiment, a kit comprises components necessary for identifying, isolating, and/or enriching a population of CD8A-positive, CD44-positive and CD103-positive CD8+ T_{RM} from a biological sample. In another aspect of the embodiment, a kit may further include positive and/or negative controls and/or instructions for identifying, isolating, and/or enriching of CD8A-positive, CD44-positive and CD103-positive CD8+ T cells by use of the kit's contents. In still other aspects of this embodiment, the kit may further include culture containers like dishes or flasks, culture medium, or any necessary buffers, factors, useful to promote cell growth.

Instructions for use may be included in the kit. “Instructions for use” typically include a tangible expression describing the technique to be employed in using the components of the kit to effect a desired outcome, such as to treat, reduce the severity of, inhibit or prevent age-related neurodegeneration in a subject. Optionally, the kit also contains other useful components, such as, measuring tools, diluents, buffers, pharmaceutically acceptable carriers, syringes or other useful paraphernalia as will be readily recognized by those of skill in the art.

Various embodiments provide that detection of CD8A-positive, CD44-positive and CD103-positive CD8+ T_{RM} or other biomarkers on CD8+ T_{RM} is performed using flow cytometry analysis based on the fluorescence-activated cell sorting (FACS) technique. A detailed standard operating procedure of FACS flow cytometry analysis is provided in Example 2.

### Methods of Using

A method of identifying a subject susceptible to or experiencing a pathological neurodegeneration, which includes detecting an increased presence of CD103-positive resident memory CD8+ T cells (CD8+ T_{RM}) in the periphery blood of the subject. Further aspect provides the subject is a human subject of an age of at least 65 years old and is detected with an increased level of CD8A-positive, CD44-positive and CD103-positive CD8+ T_{RM} in the blood.

A method is also provided of quantifying CD103-positive CD8+ T_{RM} in a subject with a memory disorder or an age-related neurodegeneration, including detecting a quantity of CD103-positive CD8+ T_{RM} cells in a biological sample from the subject. In some embodiments, the method further includes comparing the quantity of CD103-positive CD8+ T_{RM} cells to a reference value.

Various ways of detection in a biological sample are available including but not limited to flow cytometry, western blotting analysis, enzyme-linked immunosorbent assay, immunoprecipitation, UV spectrometer, chromatography, mass spectrometry, immunohistochemical staining and imaging.

A reference value in a quantification assay or method can be the value obtained from a control subject, such as a healthy subject without any symptoms of a memory disorder or neurodegeneration, or that obtained from a pool of such control subjects. In other embodiments, a reference value is the subject's own numbers of a younger age where no or few symptoms of a memory disorder is shown, for use as a reference to determine the high risk, the need for treatment thereof, or the suboptimal result of a treatment if the current value exceeds the reference value. In yet another embodiment, a reference value is the subject's own numbers prior to a treatment of a memory disorder or neurodegeneration, for use as a reference to determine the efficacy of a treatment.

A method is provided for treating, inhibiting, reducing the severity of or promoting prophylaxis of age-related cognitive decline, pathological neurodegeneration, or both in a subject in need thereof. The method includes administering to the subject a therapeutically effective amount of one or more of an inhibitor of CD103, an inhibitor of effector T cells that arise from resident memory CD8+ T cells (CD8+ T_{RM}), and an inhibitor of the molecules released by the effector T cells that arise from CD8+ T_{RM}. In some embodiments, the inhibitor of CD103 in the method is an anti-CD103 antibody, and the inhibitor of effector T cells that arise from resident memory CD8+ T cells includes an inhibitor of perforin-1 or an inhibitor of IFNγ. One aspect provides the administration reduces reaction or binding of CD8+ T_{RM} or effector T cells derived therefrom to an APP peptide, e.g., peptide SEQ ID No: 8, compared to that obtained from a control subject, such as a healthy subject without any symptoms of a memory disorder or neurodegeneration, or to that obtained from a pool of such control subjects. Another aspect provides the administration reduces reaction or binding of CD8+ T_{RM} or effector T cells derived therefrom to an APP peptide, e.g., peptide SEQ ID No: 8, compared to the subject's own numbers of a younger age where no or few symptoms of a memory disorder is shown.

A method is also provided for treating, inhibiting, reducing the severity of or promoting prophylaxis of age-related cognitive decline, pathological neurodegeneration, or both in a subject in need thereof. The method includes administering to the subject a therapeutically effective amount of a tolerogenic vaccine delivering amyloid precursor protein or a peptide fragment thereof, e.g., any of those of SEQ ID Nos: 2-8.

A method of identifying a human subject susceptible to or experiencing an age-related cognitive decline or a pathological neurodegeneration is provided, which includes detecting an increased presence of CD103+ resident memory CD8+ T cells (CD8+ T_{RM}) in a blood sample obtained from the human subject with one or more symptoms of loss of short-term or long-term memory, decreased ability to maintain focus, and decreased problem solving capacity. An aspect of the method provides the increased presence of CD103+ CD8+ T_{RM} is compared to a value obtained from one or a pool of healthy human subjects with none of the one or more symptoms. Another aspect provides the human subject is at least 65 years old, or at least 50, 55, or 60 years old.

In various embodiments, the subject in the methods is a human. In some embodiments, the human subject is at his or her middle age, or later, e.g., after 30, 35, 40, 45, 50, 55, 60, 65, 70, 75, 80, 85, 90 or 95 years old. In other embodiments, the human subject has shown previous records of CD103-positive CD8+ T_{RM} cells.

In various embodiments, an age-related cognitive loss, a pathological neurodegeneration, a memory disorder, or the like, in one or more of the above-mentioned methods and compositions includes symptoms from forgetfulness or loss of short-term or long-term memory, decreased ability to maintain focus, decreased problem solving capacity, multiple sclerosis, Parkinson's disease and Alzheimer's disease.

### An Animal Model and More

A system is provided to identify and/or screen candidate therapeutic, prophylactic and/or diagnostic agents for human cognitive decline, where the system includes CD44^{hi}CD123⁺CD127^{hi}KLRG1⁺CD103⁺ resident memory CD8+ T cell phenotype that is obtained from a rodent (e.g., mouse) animal. In some embodiments, the CD44^{hi}CD123⁺CD127^{hi}KLRG1⁺CD103⁺ resident memory CD8+ T cell phenotype is obtained by administering resident memory CD8+ T cells in a thymus-deficient mouse.

A process of identifying and/or screening a candidate agent for human therapeutics or prophylaxis of age-related neurodegeneration includes contacting the candidate agent with the CD44^{hi}CD123⁺CD127^{hi}KLRG1⁺CD103⁺ resident memory CD8+ T cells in vitro or administering the candidate agent to a model animal containing the CD44^{hi}CD123⁺CD127^{hi}KLRG1⁺CD103⁺ resident memory CD8+ T cells, to identify a reduced level of CD103-positive resident memory CD8+ T cells, a reduced level of their effector molecules, or a reduced amount of the emigration of CD8+ T cell from the periphery system to the brain of the animal.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Aberrant Resident Memory CD8+ T Cells Mediate Pathological Neurodegeneration and Age-Related Cognitive Decline

CD8+ T cell homeostatic expansion is a function of low T cell numbers, and occurs not only gradually with age, but rapidly upon injection into young T cell-deficient hosts (20, 21). This inducible phenomenon could allow unambiguous testing of the role of abnormal CD8+ T cells in diseases such as Alzheimer's disease, provided its relevance to age-related CD8+ T cell dysfunction is established. It was determined that spontaneous homeostatic induction by injection into nude mice uniformly induced CD8+ T (“hiT”) cells that exhibit molecular, phenotypic, and functional abnormalities indistinguishable from those in affected aged mice. These hiT cells localized to brain, where they ultimately promoted Alzheimer's disease-like neurodegenerative pathology, including prominent disease features missing from FAD-mutant transgenic animals. hiT cell-associated metrics were also increased in human Alzheimer's disease brain. Our study identifies an age-related immune-cellular process that overcomes mouse resistance to Alzheimer's disease-like pathology induced by aging and risk factors. These findings have important implications for modeling age-related diseases in mice, as well as for the modeling, etiology, and treatment of sporadic Alzheimer's disease.

### Materials and Methods

Animal Subjects

Female C57BL/6, B6.Foxn1 mice, and congenic and/or syngeneic knockout strains (Jackson Labs) were housed in a pathogen-free vivarium under standard conditions on a 12-h light/12-h dark cycle with food and water ad libitum. Recipient animals were 8-10 week-old female B6.Foxn1 (n>5), B6.Foxn1-AppKO (n>4), or B6.CD45.1-congenic (n>5) mice; donors were 5-8 week-old females of the same strains. Cell derivation was randomized by pooling from >5 donors per experiment. Young (8-10 wk) and aged (15 months) male and female C57BL/6 and B6.CD103-knockout mice (n=12 young; n=7-8 aged) were used to study age-related cognitive decline. Donor, recipient, and unmanipulated animals were maintained in a pathogen-free facility under the Cedars-Sinai Department of Comparative Medicine, with all breeding and genetic screening conducted at Jackson Laboratories (Bar Harbor, Me.).

Adoptive Transfer of CD8+ T Cells

Splenic CD8+ T cells from C57BL/6J female mice (5-7 weeks old) were purified using anti-CD8 immunobeads (Miltenyi Biotech, Sunnyvale, Calif.). 3×10⁶ CD8+ T cells in 50 μl of PBS were injected i.v. into female C57BL/6J or B6.Foxn1 nude hosts. Transfer efficiency into B6.Foxn1 hosts was validated by persistence of >5% CD8+ T cells within splenic lymphocytes 3 weeks after injection. The order of treatments was randomized by alternating cell and control injections between individual recipients. For all subsequent analyses, performing investigators were blinded to both group definition and anticipated outcomes.

Tissue Processing (Brain, Spleen)

Brain and spleen was harvested from PBS-perfused mice. Brains were sectioned 1 mm to the right of the longitudinal fissure (midline). Right hemispheres were flash frozen in −80° C. conditions for protein studies, followed by homogenization in Cell Lysis Buffer (Cell Signaling Technologies, Mass.), and centrifugation of nuclei. Cell lysates were separated into Triton soluble, Sarkosyl soluble and Sarkosyl insoluble fractions using sequential incubations of 10% (wt/V) salt sucrose solution and 1% (wt/v) sarkosyl Salt Sucrose Solution. Left hemispheres fixed in 4% paraformaldehyde and reserved for immunohistochemical staining. Brain Weight standardization: Upon removal of whole brain from cranium, cerebellum, brainstem, and olfactory bulbs were removed prior to weighing on a Mettler balance.

Western Blot

Triton-soluble cell lysates were electrophoretically separated on 12% Tris-HCl Precast Gels (Bio-Rad), and blotted onto 0.2 μm nitrocellulose. Membranes were blocked with BSA, incubated in sequential primary and secondary antibody dilutions for 1 hour at room temperature with >3 washes, developed with enhanced chemiluminescence substrate (GE Healthcare Biosciences; Pittsburgh, Pa.), and exposed onto Amersham Hyperfilm (GE Healthcare Biosciences; Pittsburgh, Pa.).

ELISA

Supernatant from homogenized brain tissues was used for Triton-soluble Aβ. Insoluble pellets from Triton-homogenized brain were resuspended in 10 volumes 5M Guanidine HCl 4 hr to generate Guanidine-soluble Aβ. Triton- and Guanidine-soluble samples were subjected to analysis by Soluble and Insoluble Aβ ELISA (Invitrogen, Life Technologies; Grand Island, N.Y.). Absorbance was read on a SPECTRAmax Plus384 microplate reader (Molecular Devices, Sunnyvale, Calif.) with data analyzed in Graphpad PRISM (Graphpad Software; San Diego, Calif.).

Flow Cytometry

Purified T cells stained with respective Abs were analyzed by three-color flow cytometry (FACScan II; BD Biosciences, San Jose, Calif.) to assess purity. Antibodies were incubated with whole-spleen single cell suspension in PBS with 5% FBS, on ice for 30 minutes, followed by a wash with the PBS with 5% FBS. 100,000-300,000 flow events were acquired.

Antibodies for Tissue Staining and Westerns

Free-floating brain sections (8-14 μm thick) were mounted onto slides and blocked for 1 h at RT. Sections were incubated at 4 oC overnight with primary antibody in blocking solution (Dako, Calif.). Sections were rinsed 4× in PBS, and incubated 90 min in fluorochrome- or biotin-conjugated secondary antibody, with or without curcumin (0.01% in PBS), or with ThioS alone (1% in PBS). Sections were washed, coverslipped, and mounted with ProLongGold anti-fade media with DAPI (Invitrogen). Bright-field and fluorescent images were obtained using a Zeiss AxiolmagerZ1 with CCD camera (Carl Zeiss Micro imaging). Image analysis of micrographs was performed with ImageJ (NIH). Anti-Aβ/APP antibody (ab14220, Abcam for 3-week time point; clone 4G8,Chemicon for all others) was used at 1:500 for immunohistochemistry (IHC) and 1:1000 for Western blot (WB). Anti-pTau pS199/202 antibody (Invitrogen) was used at 1:50 for IHC and 1:100 for WB, with PHFs confirmed with Phospho-PHF-tau pSer202+Thr205 Antibody (AT8), used at 1:2000 for WB. Due to marker size, pTau WB signal was normalized to that of β-actin (clone AC-74, Sigma), with GAPDH used for normalization of all other markers. Anti-GFAP (Dako) was used at 1:250 for IHC and WB. Anti-NeuN antibody (Chemicon) was used at 1:100 for IHC and WB. Anti-Iba1 (Wako, Ltd.) was used at 1:200 for IHC. Anti-CD8 (clone 53-6.72, BD Pharmingen) was used at 1:100 for IHC and 1:1000 for WB. All secondary antibodies (HRP, Alexa Flour-488, -594, -647; Invitrogen) were used at 1:200 for IHC and 1:2000 for WB. Multimer generation & use: dextramers of established epitopes for self/brain antigen (Trp-2-DCT(180-188)/H-2Kb), and/or custom APP epitopes with predicted affinities <100 nM (NetMHC version 3.4), were manufactured by Immudex.

Gallyas Silver Staining

Gallyas silver stain was used to visualize fibrillar aggregates. Free floating brain sections were placed in 5% Periodic Acid 3 min, washed twice and placed in Silver Iodide solution 1 min, followed by incubation in 0.5% Acetic Acid 5 min (2×), and rinsed with dH20. Sections were incubated in developer for ˜10 min until sections were pale brown/gray, and stopped in 0.5% acetic acid 5 min, rinsed in dH20 and mounted. Stained sections were examined by microscopy. Stained neurons were counted from CA2 of hippocampus, and their proportions within total neurons visually quantified in triplicate from entorhinal and cingulate cortex.

Neuronal Counts

Whole-number neuronal estimates were performed using the optical fractionator method with stereological software (Stereo Investigator; MBF Bioscience). Para-median sagittal serial sections spaced 50 μm apart were stained with NeuN. CA1, CA2, CA3 and other regions of interest were defined according to the Paxinos and Watson mouse brain atlas. A grid was placed randomly over the ROI, and cells counted within three-dimensional optical dissectors (50 μm 50 μm 10 μm) using a 100× objective. Within each dissector, 1 μm guard zones at the top and bottom of section surface were excluded. Estimated totals weighted by section thickness were obtained with Stereo Investigator software, yielding a coefficient of error 0.10.

Behavioral Testing

Open Field testing was performed preceding all other behavioral tests, at 3, 6, and 13 months post-cell or -control injection. Flinch-jump/Fear Conditioning freezing times were determined 6 and 11 months post-cell or -control injection. Mice were tested for SA a single time only, 12 months post-cell or -control injection. Barnes Maze testing was performed a single time only, 14 months post-cell or -control injection. The order of behavioral tests was randomized by alternating control and treatment group animal runs. Tests were begun at the same time (+/−1.5 hr) for tests run on more than one day, with early and late times alternated for inter-group randomization. In the Barnes maze, additional randomization of alternating escape compartment location between each animal per group, and between each of 3 daily training tests per animal, was employed.

Barnes Maze (BM) Test

Barnes maze is a spatial-learning task that allows subjects to use spatial cues to locate a means of escape from a mildly aversive environment (i.e. the mice are required to use spatial cues to find an escape location) Mice were assessed for their ability to learn the location of an escape box over the course of 9 days in the BM apparatus. The escape hole is constant for each mouse over the five training days. Each mouse was tested three times per day (3 trials) for 4 days, followed by no testing for 2 days, and re-testing on day 7. A 35-60 minute inter-trial interval separates each trial. Each trial began by placing one mouse inside a start box with a bottomless cube positioned centrally on the maze. After 30 seconds, the start box was lifted and the mouse was released from the start box to find the escape hole. Two fluorescent lights located on the ceiling or high above illuminate the testing room. Each trial lasted up to 4 min or until the mouse entered the escape box. The experimenter guided mice that failed to find the escape hole within 4 min, to the correct hole after each training test. Once the mouse entered the escape box, it was allowed to remain in the box for 1 min. Following the 7th day of testing, and never on the same day, mice were tested an additional two-days, in which the escape box was placed in the reverse position on day 8, and replaced in the original position on day 9. The same exact testing procedure was applied to all mice in all groups. The maze and all compartments were cleaned thoroughly with isopropyl alcohol to remove any olfactory cues after each trial, and prior to each day of testing.

Y-Maze Spontaneous Alternation (SA) Test

Y-Maze Alternation Test is used to assess working memory. Spontaneous alternation was measured by individually placing animals in one arm of a symmetric Y-maze made of opaque black acrylic (arms: 40 cm long, 4 cm wide; walls: 30 cm tall), and the sequence of arm entries and total number of entries recorded over a period of 8 min. Mice were tested for SA a single time only.

Flinch-Jump/Fear Conditioning Tests

It was first determined that there were no significant differences in the nociceptive threshold (pain sensitivity) across treatment groups using the Flinch-Jump Test. Pavlovian Fear Conditioning was then used to assess learning and memory regarding aversive events. The apparatus (Freeze Monitor™, San Diego Instruments, San Diego, Calif.) consisted of a Plexiglas box (25.4×25.4×31.75 cm high) with a stainless steel grid floor. An acoustic stimulus unit is located on top of the box, and the box is ringed with photo beams and optical sensors. The optical sensors were connected to a computer by way of an input matrix, and breaks in the photo beams ere automatically recorded. For testing, on Day 1 individual mice were placed into the test box, and allowed to habituate for 3 minutes. At 3 minutes a tone was presented for 30 sec. 30 sec after termination of the tone, a 0.5 sec foot shock (intensity=mean jump threshold for the treatment group determined by the Flinch-Jump Test) was delivered. The mouse was then removed from the box and returned to its home cage for 2 minutes. The chamber was cleaned and the animal returned to the chamber where the procedure is repeated. The freeze monitor apparatus recorded freezing times throughout the procedure (absence of movement for 5+ seconds, resulting in no beam breaks). On Day 2, context retrieval is determined by placing the mouse into the same test box where it previously received tone and foot shocks, but here the tone and foot shocks were not presented. Freezing time was measured over a 10 min period. On Day 3, cue conditioning was measured after inserting a triangular, plexiglass box into the test box. The mouse was placed into the triangular chamber where they had not previously received tone or foot shocks, but after 1 min the auditory tone was delivered for 30 sec and freezing time measured for 10 min. All data from Flinch-Jump and Fear Conditioning Tests was normalized, first within each group to the average of the initial two tests in training on day 1, and then within all experimental groups to the average contextual or cue values of PBS controls, expressed as percent of control, and analyzed by ANOVA, followed where appropriate by Newman-Keuls tests to detect differences among treatment groups.

Open Field Test

The test was carried out in an Open Field apparatus made up of an open topped, clear Plexiglas box, measuring 16″×16″ and 15″ high. Two rings of photobeams and optical sensors surrounded the box. The optical sensors were connected to a computer by way of an input matrix. Each mouse was placed into the box, and breaks in the beam interruptions automatically recorded and used as a measure of locomotor activity. Each mouse was tested in the box for a period of 30 minutes.

Statistical Analysis

Quantification and stereological counting procedure for cell numbers or area (μm2) of Amyloid beta plaque, GFAP+, Iba1+ or Perforin1+ cells were analyzed in six to eight coronal sections from each individual, at 150-μm intervals (unless otherwise indicated), covering 900-1200 μm of the hippocampal and cortical areas. Specific fluorescence signal was captured with the same exposure time for each image and optical sections from each field of the specimen were imported into NIH Image J and analyzed as above. GraphPad Prism (version 5.0b; San Diego, Calif., USA) was used to analyze the data using ANOVA and T-Tests with Welch's correction (no assumption of equal variance). In all histograms, average+SEM is depicted.

Sample sizes for PrfKO-CD8 and Ifn KO-CD8 groups were calculated a priori for each metric using means and standard deviations of PBS and wt-CD8 groups for anticipated effect sizes, with alpha 0.05, and >95 confidence. Calculated n plus >1 were then used for PrfKO-CD8 and IfnγKO-CD8 groups.

Pre-determined exclusions included sections or samples with no discernible background signal, and values within each group >2 standard deviations above or below the median/group. Subject numbers and methods of reagent validation are shown in Table S1.

Study Approval

All animal procedures were approved prior to performance by the Cedars-Sinai Institutional Animal Care and Use Committee. The Cedars-Sinai Institutional Review Board designated the analysis of de-identified human brain specimens from UC Davis exempt from committee review. Brain specimens were collected, stored, and disseminated with prior approval by the UC Davis Medical Center Institutional Review Board.

### Results

Generation of “hiT” Cells in Nude Mice

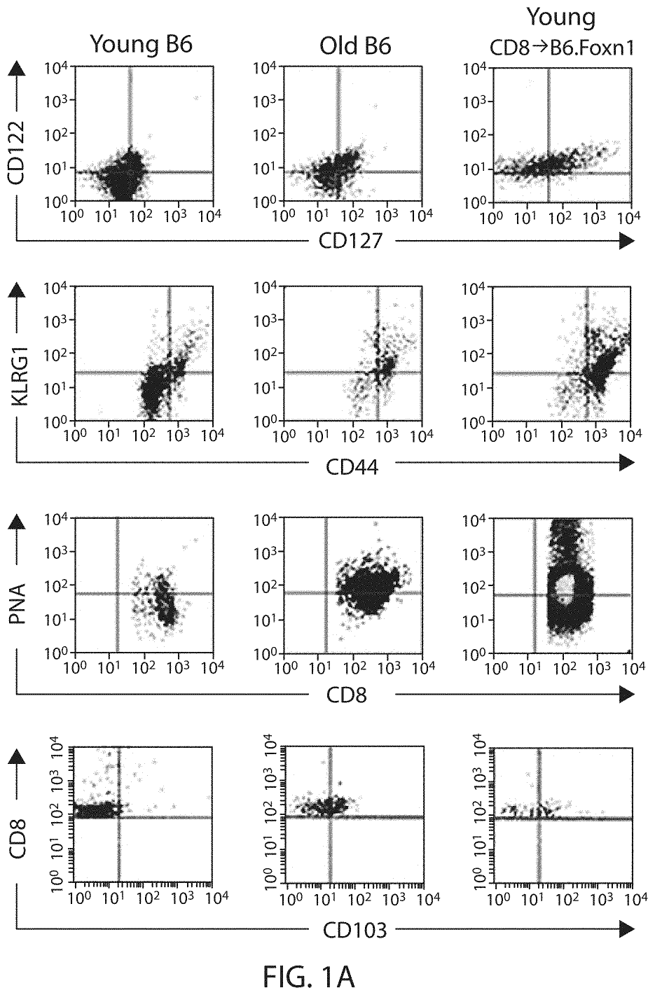

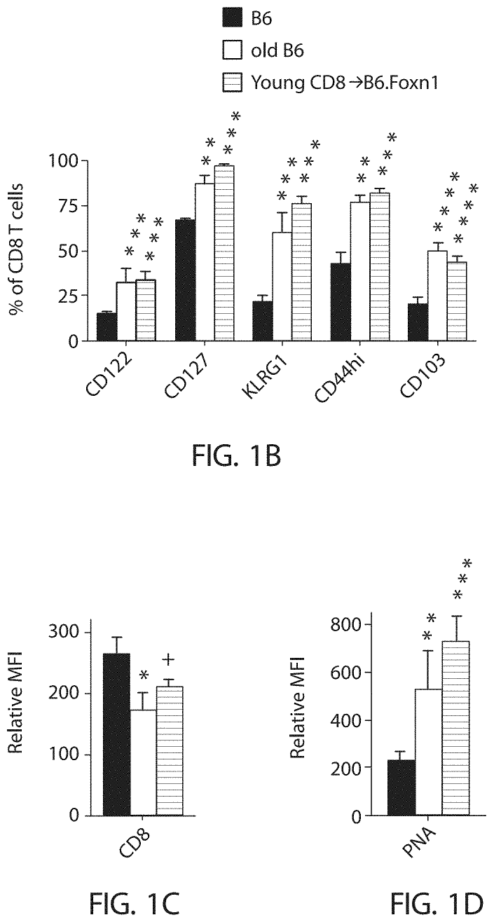

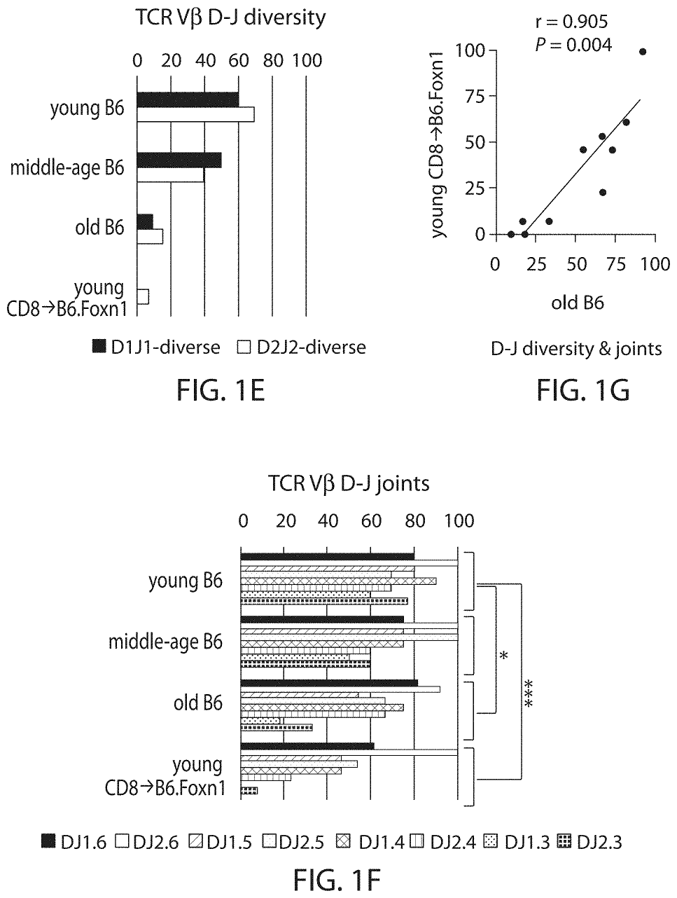

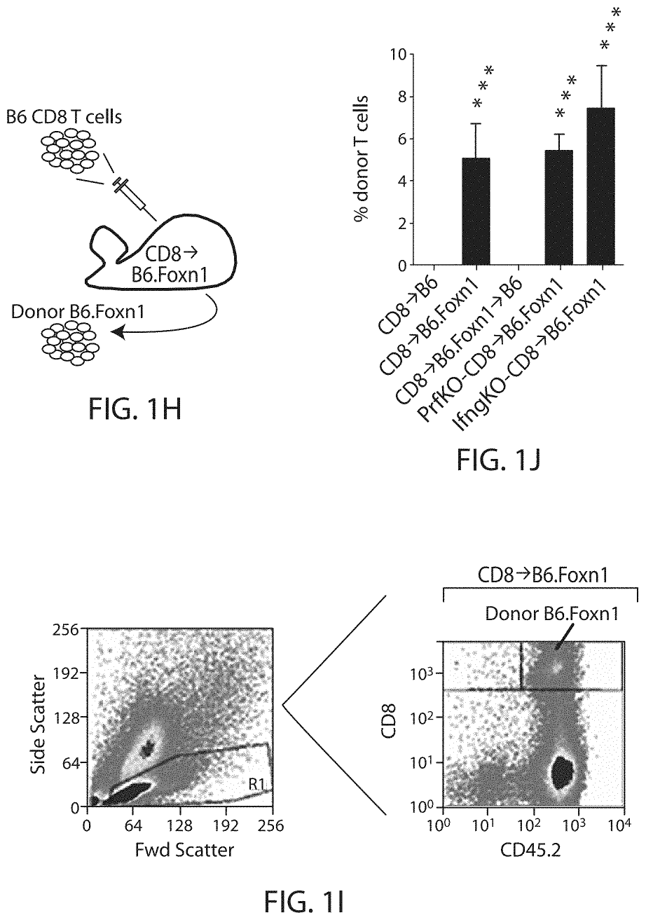

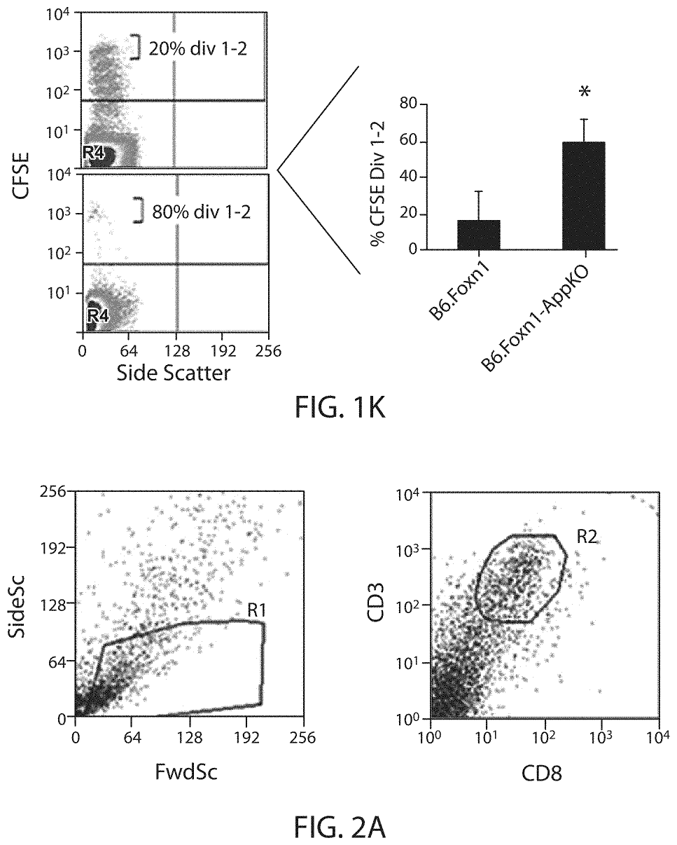

CD8+ T cells from young (<9 wk) C57BL/B6 (B6) donors were injected into B6.Foxn1 recipients, and subjected to phenotypic analysis (FIGS. 1A, 1H and 1I). Donor CD8+ T cells rapidly expanded in blood of young B6.Foxn1 recipients within 3 days, where they remained long-term (FIGS. 1J and 1K). CD8+ T cells serially transferred from nude into wild-type B6 or B6.CD45.2-congenic [B6^{(Cg)}] hosts did not expand further (FIGS. 1I and 1K). Analysis of homeostatically-induced donor CD8+ T cells (“hiT” cells) in B6.Foxn1 hosts revealed a surface marker profile identical to CD8+ T cells that have undergone clonal expansion in aged mice (CD122^{hi}, CD127^{hi}, CD44^{hi}, KLRG1^{hi}, PNA^{hi}, CD8^{lo}, CD103⁺; FIGS. 1A-1D). A similar phenotype is found on CD8+ T cell clonal expansions in aging humans. Accordingly, CFSE-labeled CD8+ T cells exhibited the laddered dye dilution and population enlargement typical of homeostatic expansion (FIG. 1K). This did not, however, occur in nude mice lacking the amyloid precursor protein (APP) gene (B6.Foxn1xAppKO mice), indicating that rapid homeostatic expansion may be dependent on reactivity to APP.

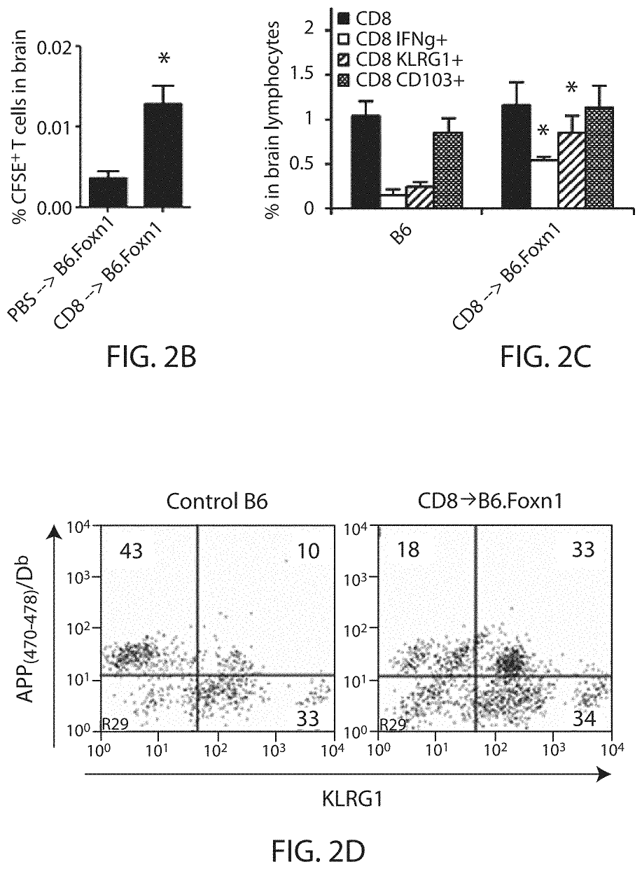

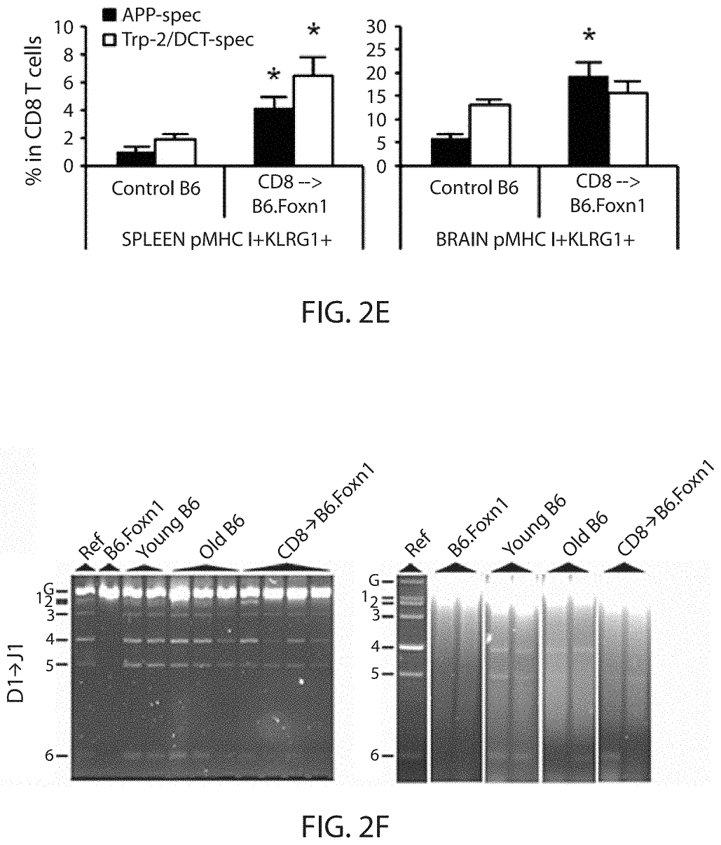

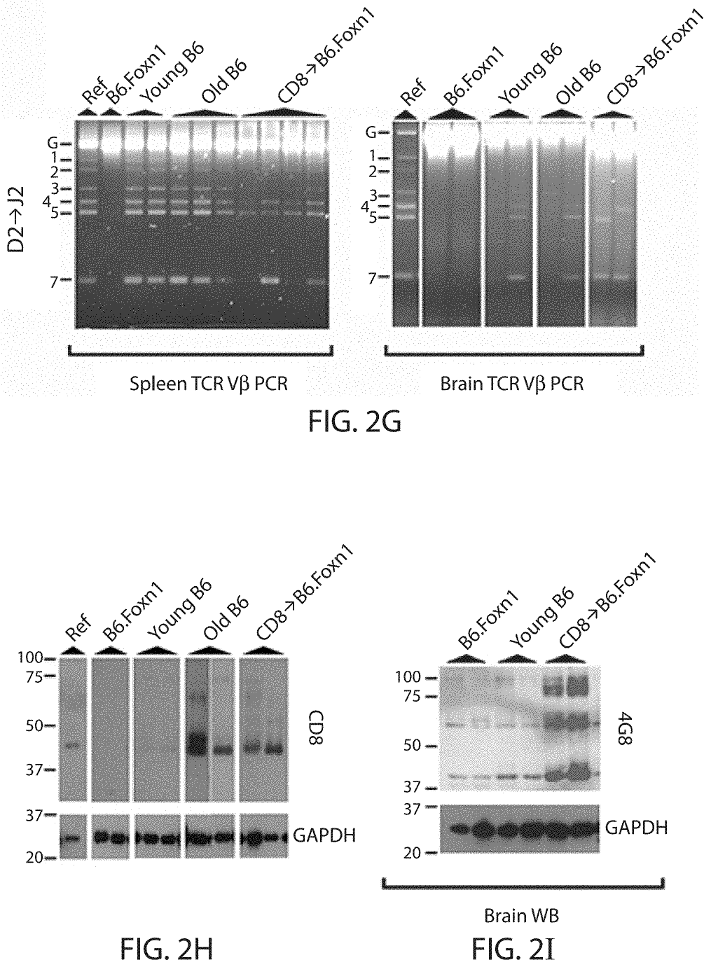

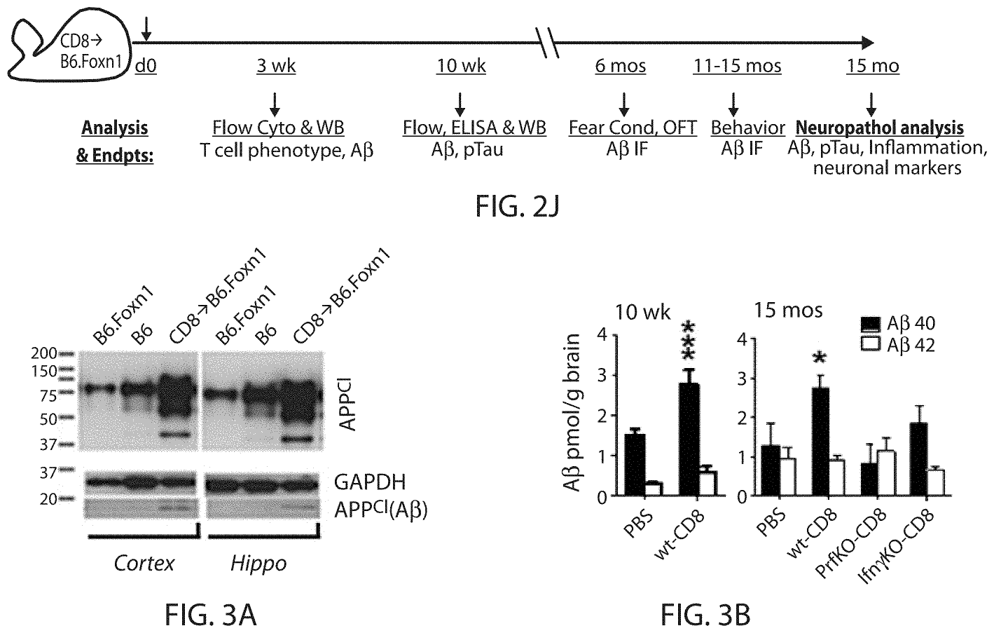

To examine clonality of hiT cells, we analyzed variable region D→J rearrangements in T Cell Receptor beta gene segments by PCR. Consistent with previous reports, peripheral T cells in wild-type mice aged 12 months showed no evidence of clonal skewing in TCRVβ, whereas those injected into nude recipients exhibited both D1→J1 and D2→J2 clonal skewing after just 10 weeks (FIGS. 2F and 2G). Importantly, D1→J1 and D2→J2 clonal skewing was also evident in brains of young nude mice injected with CD8+ T cells, in contrast to the diverse D→J usage seen in young wild-type mice (FIGS. 1E and 1F). This pattern was most closely resembled D→J usage in brains of old-aged mice.

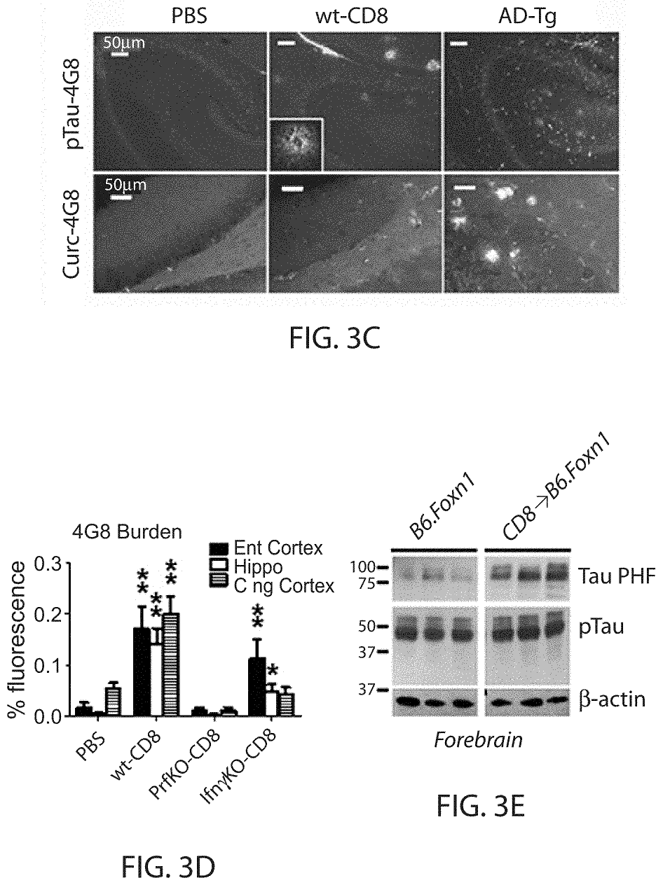

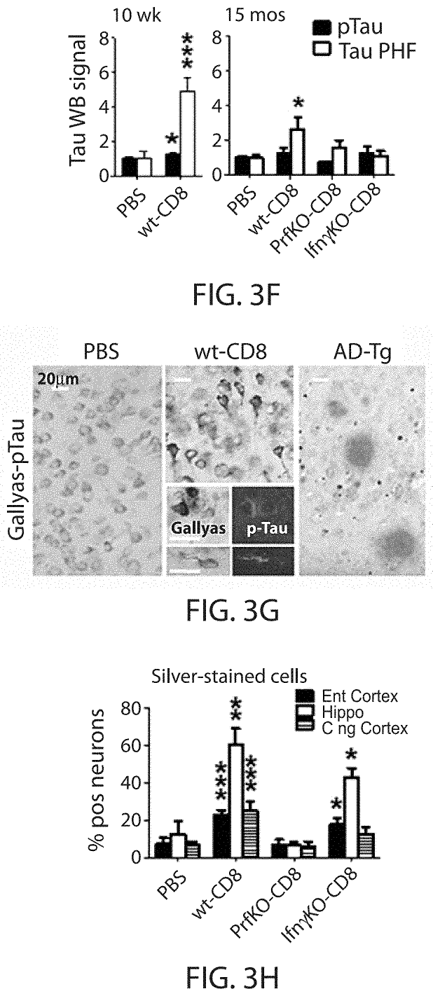

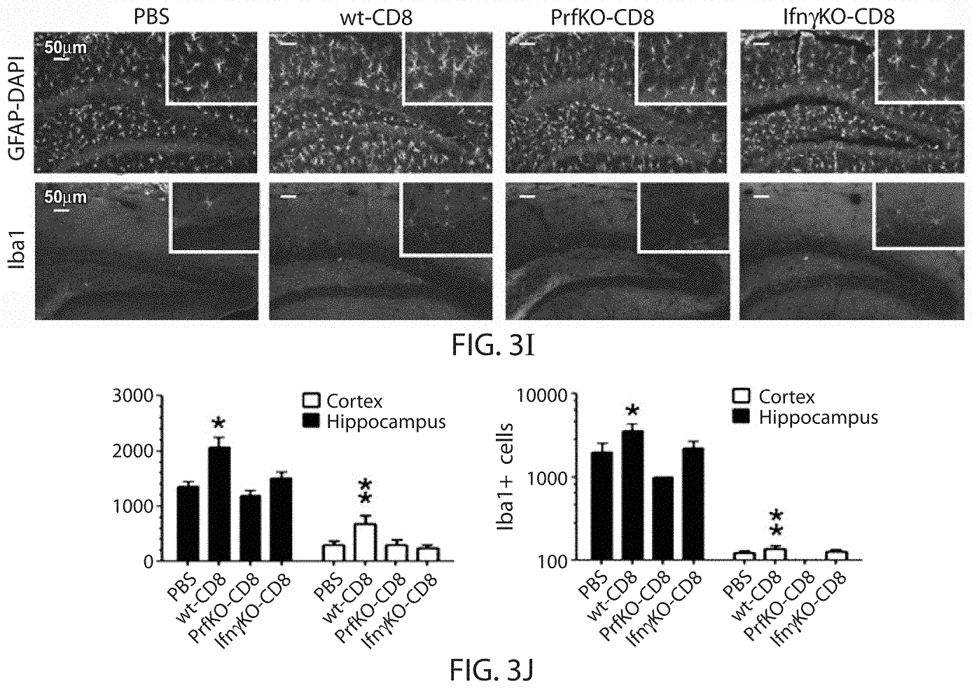

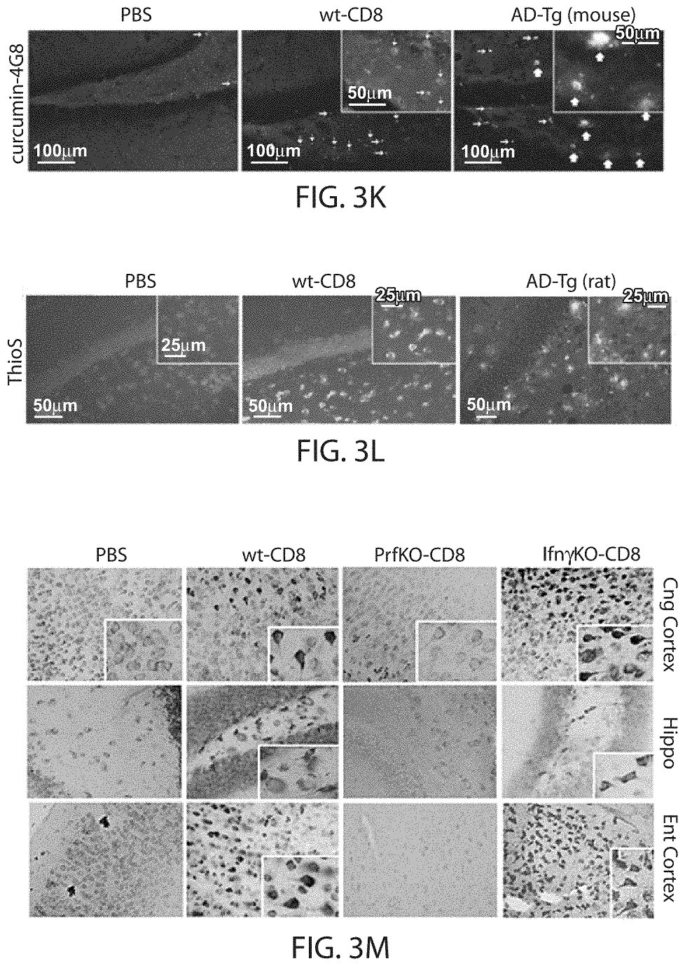

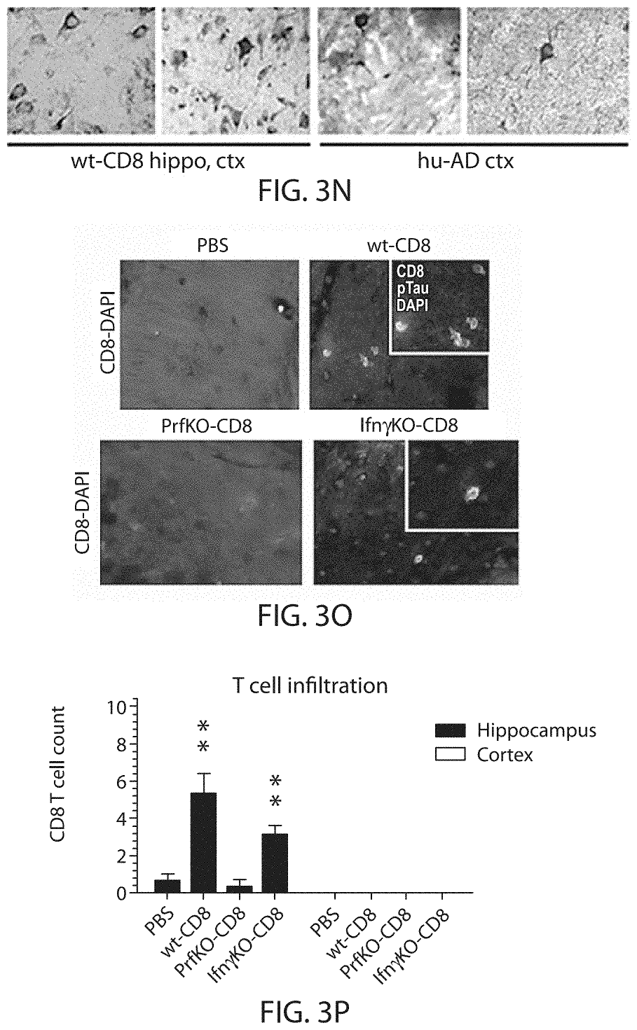

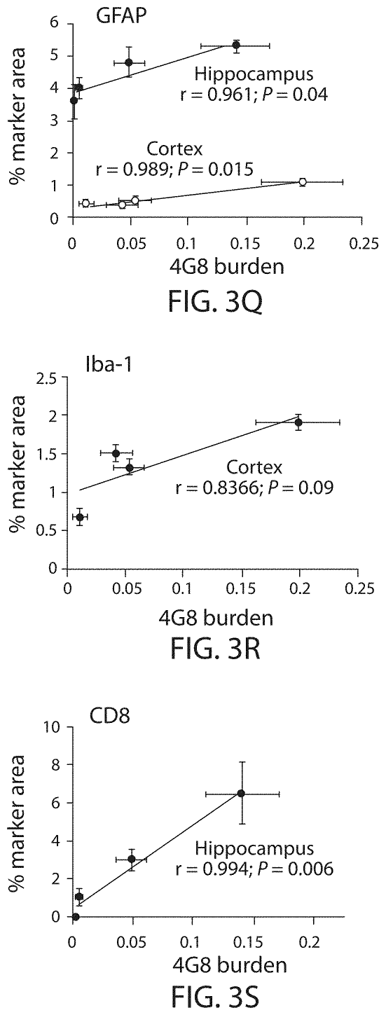

CFSE-labeled donor CD8+ T cells were increased in B6.Foxn1 in brain parenchyma three days after i.v. injection, directly confirming rapid homing of hiT cells to brain (FIGS. 2A and 2B). Total CD8+ T cells were only marginally increased 10 weeks after injection in B6.Foxn1 relative to wild-type B6 by flow cytometry (FIG. 2C), but increased CD8 protein on Westerns was evident at this time point, suggesting increased cellular influx without an increase in live cells (FIG. 2H). Indeed, IFNγ⁺ and KLRG1⁺ CD8+ T cells that retained CD103 expression were both significantly increased in nude recipients' brains by flow cytometry at this time point, indicating a qualitative but not quantitative change in CD8+ T cells within brain (FIG. 2C). KLRG1⁺ CD8+ T cells in peripheral blood were reactive to MHC I-restricted antigens, including Tyrosinase-related Protein-2/Dopachrome Tautamerase (Trp-2/DCT) and APP, but only the latter were significantly increased in brain (FIGS. 2D and 2E). Thus, hiT cells reactive to an APP epitope selectively accumulated in nude brains, leading us to analyze APP-related pathology (FIG. 3K).

Aβ and Neurofibrillary Deposition

Detergent-soluble APP and derivative cleavage products (APP^{Cl}) were increased in dissected cortex and hippocampus of B6.Foxn1 hosts 3 and 10 weeks after i.v. CD8+ T cell injection by Western blot (FIGS. 3A and 2I). Aβ1-40 was increased 2.5 months later by ELISA and remained so after 15 months (FIG. 3B), with increased Aβ on vasculature observed at 6 months (FIGS. 3L and 3M). Diffuse plaques and increased Aβ1-40 were detected in hippocampus, entorhinal cortex and cingulate cortex of B6.Foxn1 recipients injected with wild-type CD8+ T cells (wt-CD8 group mice) 15 months later (FIGS. 3C and 3N). Unlike mice expressing familial gene mutations found in human Alzheimer's disease, however, Aβ1-42 was not significantly altered, and plaques were mainly diffuse in hiT-bearing nude mice, with little co-staining by curcumin or ThioS (FIGS. 3C and 3O). Thus, amyloidopathy in hiT-bearing nude mice differed from that seen in ADtg mouse models.

Curcumin and ThioS stained cells within dentate gyms of wt-CD8 group mice 6 months after T cell injection (FIG. 3K). Similar structures were not observed in aged ADtg mice (FIG. 3K), or ADtg rats that exhibit tau paired-helical filaments (FIG. 3L). This indicated that hiT-bearing nude mice might harbor fibrillar inclusions of hyper-phosphorylated tau protein in neurons. Accordingly, Triton-soluble pTau was increased about 30%, and larger Tau PHFs increased nearly 5-fold, in wt-CD8 brains 10 weeks after injection (FIGS. 3E, 3F). While the increase in pTau was not sustained, Tau PHF remained 2.5-fold higher than controls 15 months after injection (FIG. 3F). Most intriguingly, silver-stained cells were increased in hippocampus, entorhinal cortex, and cingulate cortex of wt-CD8 group mice at this time point as well (FIG. 3G, 3H). Sequential silver-immunofluorescence staining revealed that these arose from nucleated pTau⁺ neurons, but non-nucleated “ghost tangles” were not seen (FIGS. 3G, 3M and 3N). Simultaneously stained ADtg mouse brain exhibited silver-stained plaques alone (Tg2576 mice; FIG. 3G), confirming silver-stained cells only in hiT-bearing mice. These data indicated that hiT cells promote the coordinated deposition of parenchymal Aβ40, diffuse plaques, and fibrillary inclusions in live neurons.

Immune and Neuroinflammatory Infiltration

Although not apparent by forebrain flow cytometry, numbers of CD8+ T cells were significantly elevated in hippocampal sections of wt-CD8 group mice 15 months after injection, which occasionally interacted with pTau⁺ neurons (FIGS. 3O and 3P). CD8+ T cell counts were not elevated outside of hippocampus. Cortical and hippocampal Iba1⁺ microglia, and activated GFAP⁺ astrocytes were also significantly elevated in the wt-CD8 group mice relative to controls (FIG. 3I, J). Aβ plaque burden correlated more strongly with hippocampal CD8+ T cell numbers than with cortical or hippocampal astrogliosis, consistent with a strong impact of T cells on amyloidopathy (FIGS. 3Q, 3R and 3S).

Neuronal Loss & Cerebral Atrophy

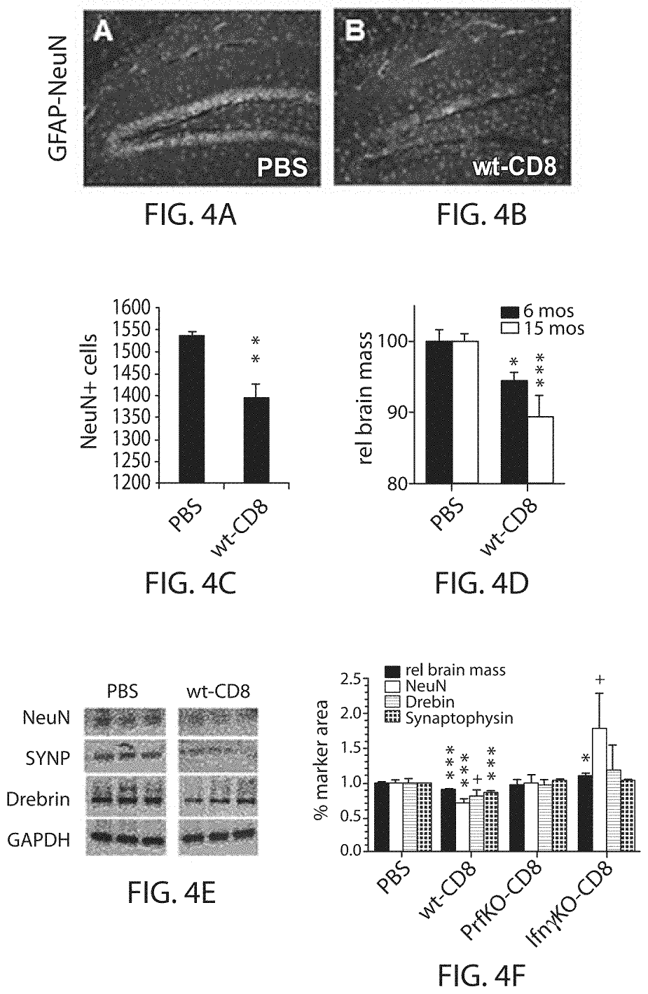

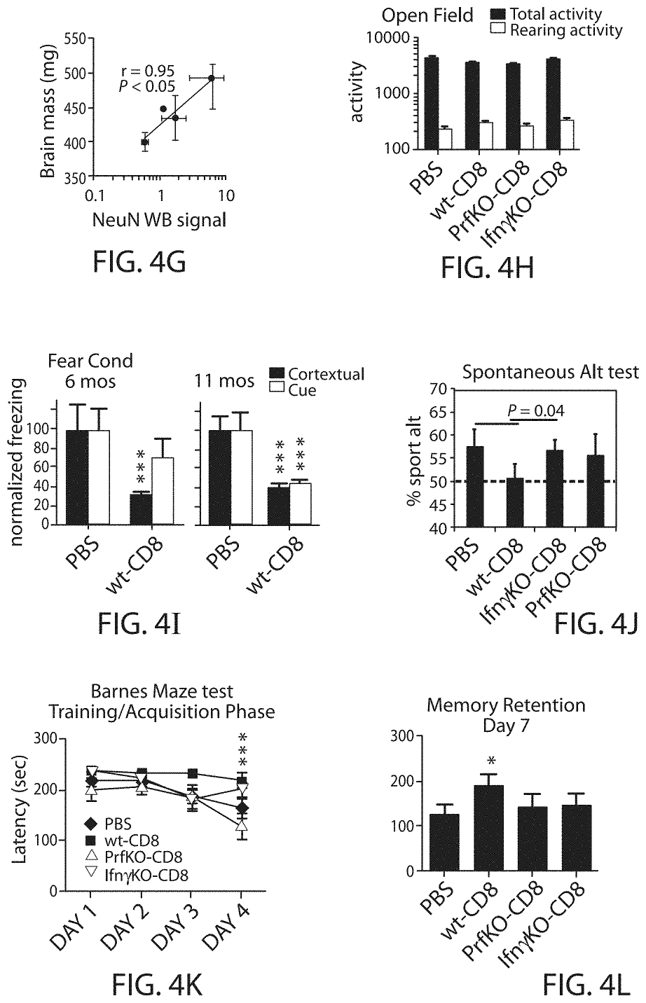

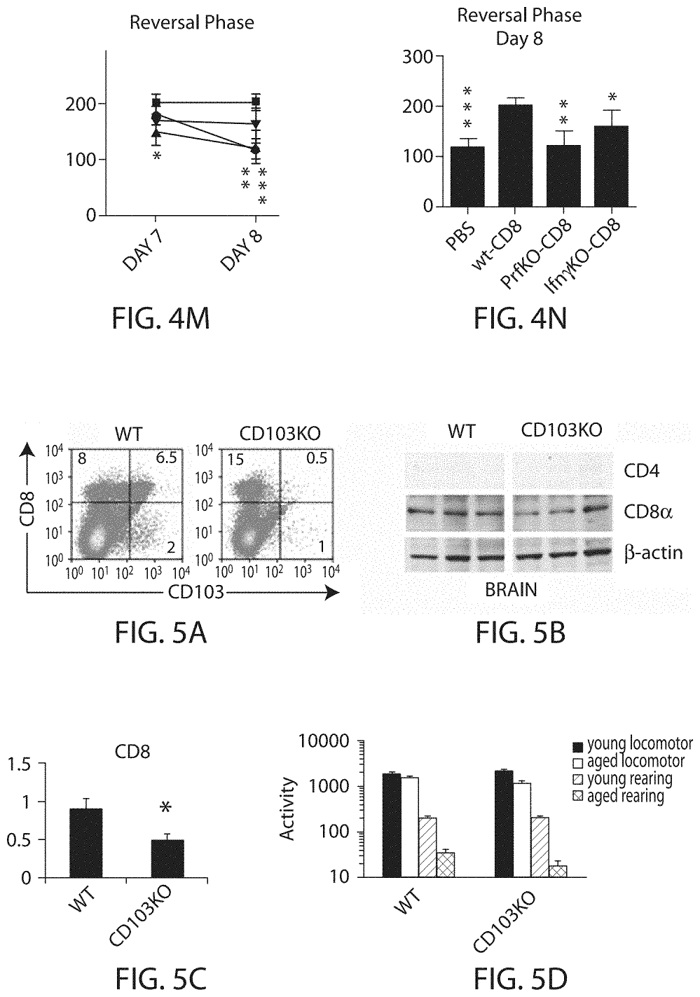

NeuN⁺ cell counts in CA2 were decreased in wt-CD8 group mice relative to controls 15 months after T cell injection (FIGS. 4A-4C). Moreover, brain mass was decreased 5% in wt-CD8 group 6 months after T cell injection, progressing to a 10% decrease at 15 months (FIG. 4D). Significant neuronal and synaptic loss in the wt-CD8 group was confirmed by NeuN, Drebrin, and Synaptophysin signal decreases on Western blot, which each exhibited roughly 10% decrease at 15 months post-injection (FIGS. 4E and 4F). NeuN Western signal correlated significantly with brain mass among treatment groups, indicating that brain atrophy reflected neuronal loss (FIG. 4G).

Severe Cognitive Impairment

Overall motor and rearing activity was not significantly different between treatment and control groups of nude recipients 3, 6, or 13 months after T cell injection (FIG. 4H). In contrast, Fear Conditioning response to contextual learning was specifically reduced in wt-CD8 6 months after T cell injection, with both contextual and cued learning impaired at 11 months (FIG. 4I). These results suggest that the cognitive impairment in the wt-CD8 group was localized to hippocampal function early (required for contextual FC), but progresses to impair both hippocampal and amygdala function later (required for cued FC). A similar pattern of progressive cognitive loss occurs in human Alzheimer's disease. Contextual learning performance at 6 months was also correlated with brain mass, indicating its relationship to neurodegeneration.

Spontaneous Alternation was measured 12 months post-injection to independently confirm cognitive deficits. This test is based on the preference of mice to alternately explore two alleys, and requires remembering the alley previously entered. The lowest possible score of 50% indicates random alley choice, reflecting either short-term memory absence, or lack of preference. Control PBS group SA was 55-56%, comparable to published wild-type values (27), but the wt-CD8 group's was 50% (FIG. 4J). To test if this reflected loss of memory or preference, we conducted the Barnes Maze test at 14 months, a definitive measure of hippocampus-dependent memory and learning. Wt-CD8 group nude mice showed no improvement in learning the maze over the initial 4-day training period, whereas all other groups exhibited substantial improvement (FIG. 4K). Given this initial deficit, wt-CD8 mice were expectedly impaired on the memory retention and reversal learning phases of the maze as well (FIG. 4L-4N). As with Fear-Conditioning, there was a significant correlation between Barnes Maze performance and brain mass. Thus, wt-CD8 group nude mice exhibited progressive, severe and lasting impairment of learning and memory, without overt motor deficits.

I further accessed whether Barnes Maze performance was associated with elevated Tau and/or Aβ metrics. Poor performance on Barnes Maze (total latency time lower than median =BM^{lo}) exhibited a non-significant association with increased soluble pTau, with a significant association with increased Tau PHFs on Westerns. By contrast, poor maze performance was not significantly associated with either Triton-soluble or GuanidineHCl-soluble Aβ40/Aβ42 by ELISA. Thus, tauopathy reflected cognitive impairment better than amyloidopathy in wt-CD8 group nude mice, as has been reported in human Alzheimer's disease.

Cellular Mechanism of hiT Cell-Mediated Neurodegenerative Pathology

To determine the mechanism(s) involved in hiT cell-mediated neurodegenerative pathology, B6.Foxn1 mice were injected with CD8+ T cells from knockout donors deficient for Perforin1 or IFNγ, key effectors of T cell lytic and proinflammatory activities, respectively. CD8⁺ T cells deficient in either gene (Prf1 and Ifnγ, respectively) expanded comparably as wild-type in B6.Foxn1 recipients (FIG. 1J), consistent with previous studies. Nevertheless, neither Perforin1-deficient nor Ifnγ-deficient CD8+ T cell recipients (PrfKO-CD8 and IfnγKO-CD8 groups, respectively) exhibited increases in soluble Aβ or pTau/PHFs at any time point (FIG. 3B, 3F). IfnγKO-CD8 group mice did, however, exhibit plaque and silver-stained cell accumulation in hippocampus and entorhinal cortex that was only slightly diminished relative to wt-CD8 group, but which did not extend into cingulate cortex (FIG. 3D, 3H). IfnγKO-CD8 group mice also exhibited reduced astrogliosis and microgliosis (FIG. 3I, 3J), but unlike the PrfKO-CD8 group, CD8+ T cells were present in brain in significant numbers 15 months after their injection (FIG. 3O, 3P). Unexpectedly, the IfnγKO-CD8 group also exhibited significant increases in both brain mass and NeuN⁺ cells at 15 months post-injection. Finally, neither PrfKO-CD8 nor IfnγKO-CD8 group mice exhibited significantly impaired cognition at 11-15 months. Thus, PrfKO-CD8 group mice showed no evidence of pathophysiology by any measure, including elevated CD8+ T cells in brain, whereas IfnγKO-CD8 group mice retained some molecular pathophysiology without evidence of neurodegeneration or cognitive decline.

CD8+ T_{RM} in Age-Related Cognitive Decline

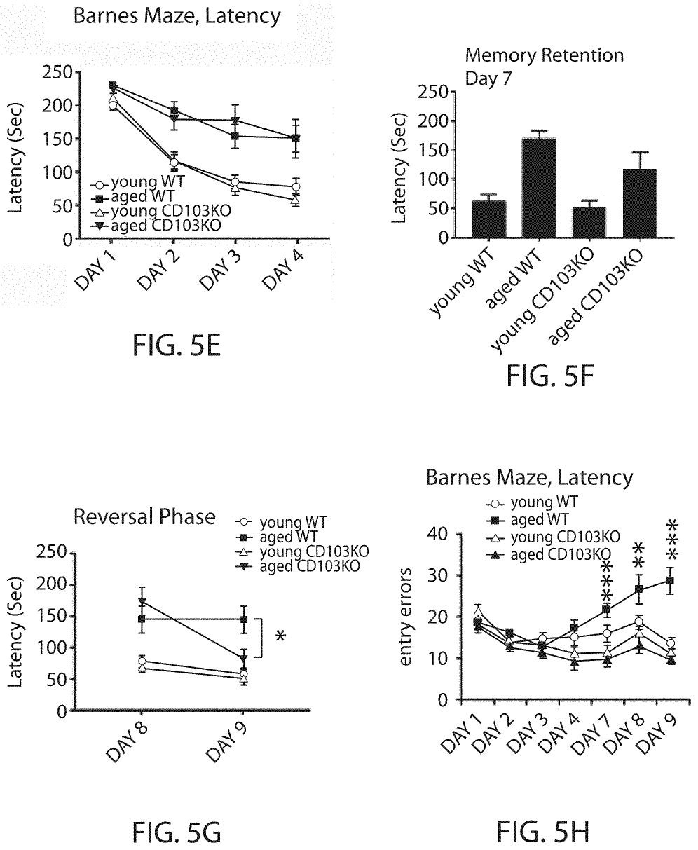

Although CD8+ T_{RM} induced cognitive neuropathology in nude mice, it was unclear whether they mediated age-associated neurodeficits in immune-competent mice. CD103 characterizes CD8+ T_{RM} that increase in aged mouse brain, and its expression is important for T_{RM} homing to brain. It is verified that genetic deficiency of CD103 impacted primarily CD8+ T cells (FIG. 5A), and diminished brain CD8 content (FIG. 5B, 5C). Young and aged CD103-deficient mice performed similarly to wild-type counterparts in the training phase of the Barnes Maze, despite reduced locomotor activity with aging (FIG. 5D, 5E). By contrast, aged CD103-deficient mice performed slightly better in memory retention and reversal phases (FIG. 5F, 5G), and made markedly fewer errors in the Barnes maze, reversing the documented age-related difference in this strain of mice (FIG. 5H). Thus, CD103 deficiency protected aged mice from age-related cognitive decline. As CD103 deficiency impacts primarily CD8+ T cells, and are the only CD103⁺ cells to increase with aging within or outside the brain, this corroborates the involvement of CD103⁺ CD8⁺ T_{RM} in cognitive decline during aging. Because age-related cognitive decline is a strong predictor of future neurodegenerative pathology, this further links CD8+ T_{RM} to pathological dementia such as AD.

hiT Cell Phenotype, Effector Protein, and Specificity in Human Alzheimer's Disease Brain

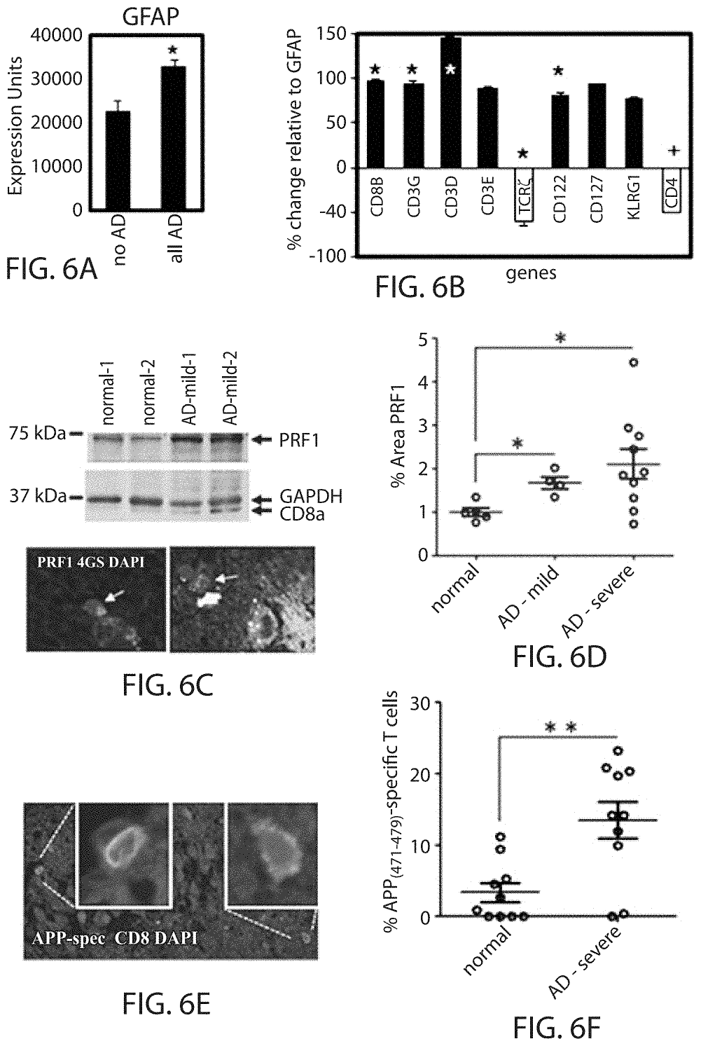

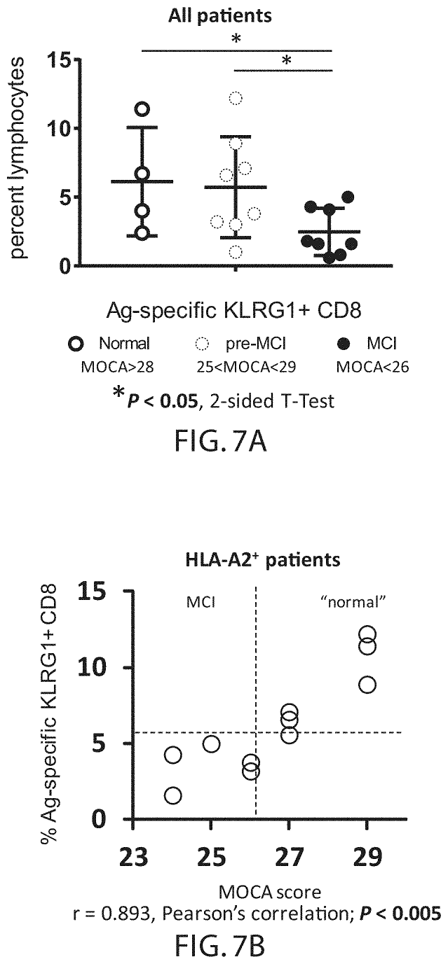

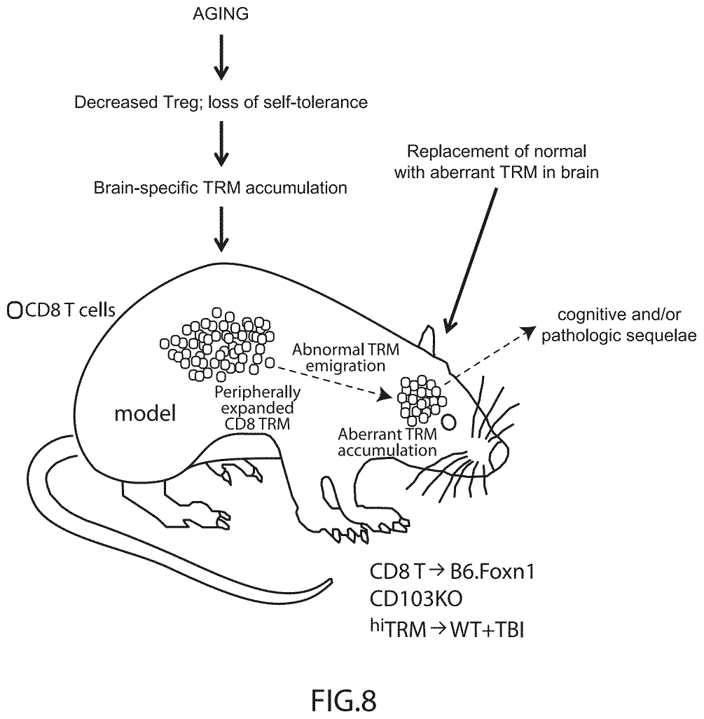

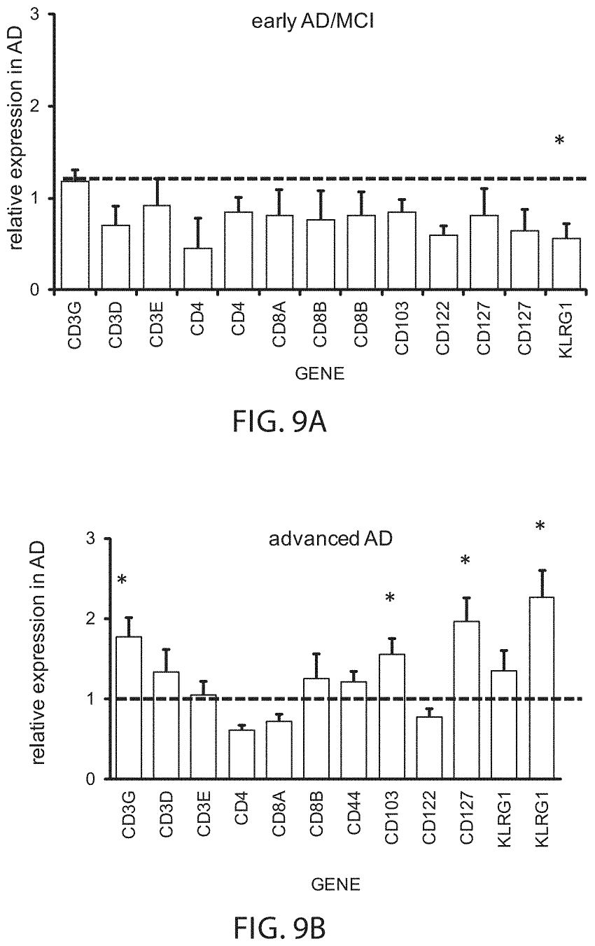

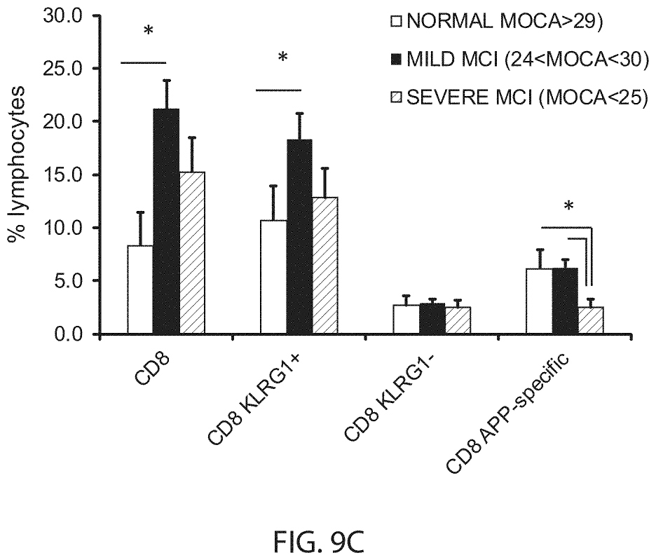

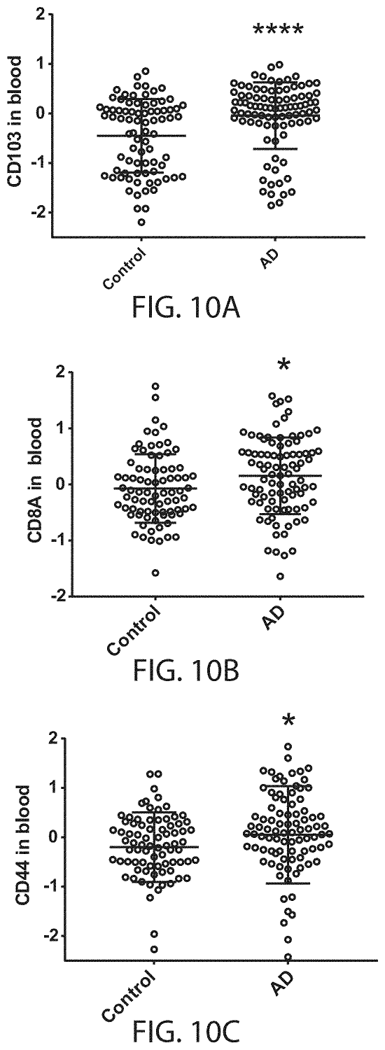

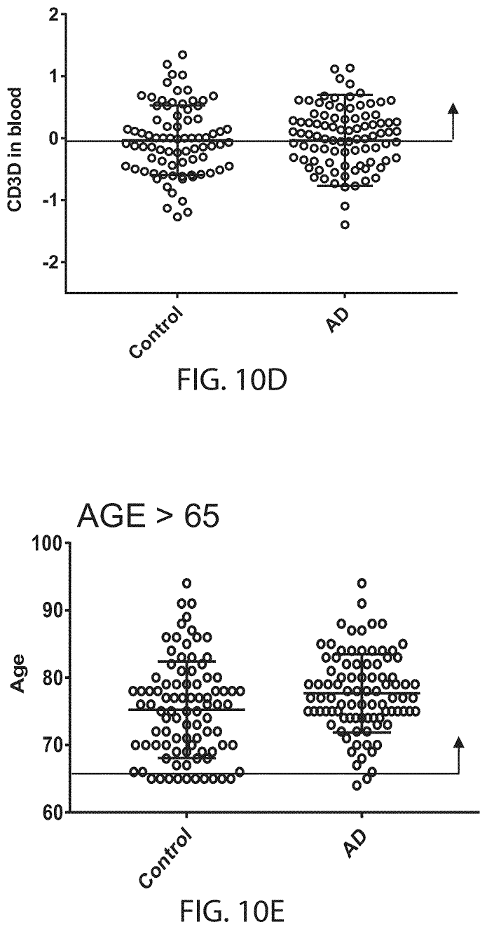

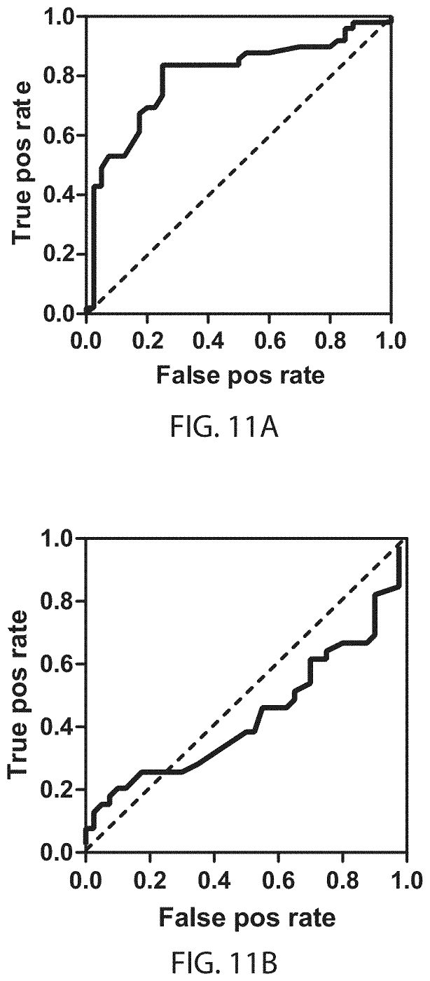

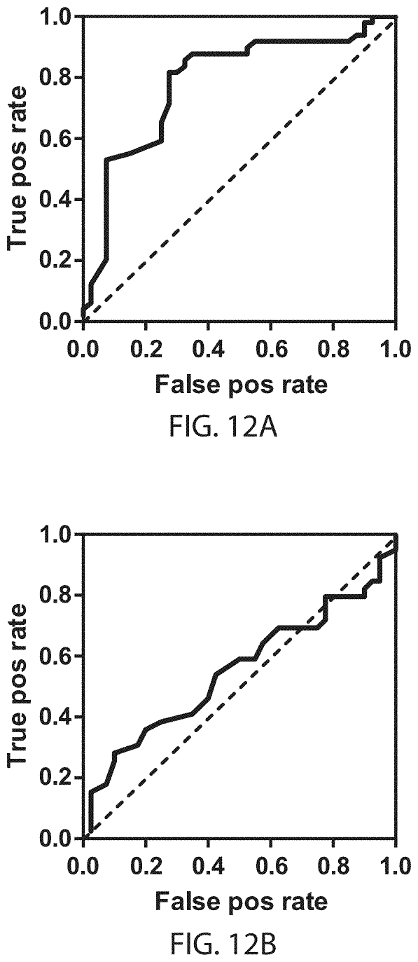

To examine possible relevance of hiT cells to human Alzheimer's disease, I focused on Perforin1 and CD8 in the diseased brain, as IFNγ is already known to be associated with disease risk. Western analysis rendered the expected antibody specificities (68-75 kDa Prf1; 33-35 kDa CD8α), with anti-Prf1 staining lymphocytic nuclei with the expected punctate pattern (FIG. 6C). Perforin1 and CD8 Western signals were correlated (n=6; r=0.8155, P=0.048), and both were increased in cortex of patients with mild, but not severe Alzheimer's disease, with Perforin1 reaching statistical significance (FIG. 6C). The ratio of Perforin1:CD8 signal was also significantly increased in severe Alzheimer's disease brain, consistent with long-term qualitative alteration of lytic lymphocyte composition as observed in hiT cell-bearing mice (FIG. 6B). To further examine this, pHLA-A2 multimers were generated to a human T cell epitope analogous to the one expanded in hiT cell-bearing mice [APP₍₄₇₁₋₄₇₉₎]. Hippocampal sections from severe Alzheimer's disease and normal aging patients with this or control multimer plus anti-CD8 were stained to quantify the proportion of epitope-reactive CD8+ T cells. Subtracting negative control staining, CD8+ T cell reactivity to APP₍₄₇₁₋₄₇₉₎ was significantly elevated in disease (FIG. 6E, 6F; P=0.002). As in hiT cell-bearing mice, total CD8+ T cell levels were slightly but not significantly elevated in disease brain (n=10; 1.6±0.29 vs. 2.3±0.55, P=0.31). Thus, APP-reactive CD8+ T cells were increased in severe Alzheimer's disease brain, similar to brains of hiT cell-bearing nude mice.

The following table lists the group numbers and validation. Validation: WB=Western blot; morph=expected morphology obtained on tissue staining; WB(absorb)=expected positive signal by Western blot with negative antigen-absorbed control; huAD=additional expected morphology obtained on brain tissue from human AD patients; co-staining=stained with 2^{nd} cell-type-specific reagent, (anti-CD8 antibody).

| | | Brain/ | | | | | |
|---|---|---|---|---|---|---|---|
| | | Region/ | | | | | |
| HOST | ANALYSIS | Method | EXPERIMENTAL GROUP “n” | | | | VALIDATION |
| | | | PBS | wt-CD8 | PrfKO-CD8 | IfnyKO-CD8 | |
| B6.Foxn1 | CD8 | Cortex | 10 | 11 | 9 | 6 | WB, IHC |
| | | Hippo | 11 | 12 | 3 | 6 | morphology |
| | GFAP | Cortex | 10 | 19 | 10 | 6 | WB, IHC |
| | | Hippo | 9 | 14 | 7 | 6 | morphology |
| | Ibal | Cortex | 3 | 5 | 3 | 5 | IHC morphology |
| | | Hippo | 4 | 4 | 3 | 3 | |
| | Aβ (10 wk) | 1-40 | 4 | 9 | NA | NA | NA |
| | | 1-42 | 4 | 9 | NA | NA | |
| | Aβ (15 mos) | 1-40 | 4 | 7 | 3 | 3 | |
| | | 1-42 | 8 | 14 | 6 | 6 | |
| | 4G8 IHC | Cng ctx | 7 | 11 | 6 | 8 | WB (absorbed), |
| | | Hippo | 6 | 11 | 5 | 8 | IHC morphology, |
| | | Ent ctx | 4 | 10 | 6 | 8 | huAD IHC |
| | pTau/PHF | 10 wk | 7 | 11 | NA | NA | WB, |
| | | 15 mos | 4 | 7 | 6 | 5 | IHC morphology, |
| | | | | | | | huAD IHC |
| | Gallyas | Cng ctx | 7 | 15 | 5 | 9 | IHC morphology, |
| | | Hippo | 7 | 13 | 6 | 19 | huAD IHC |
| | | Ent ctx | 19 | 19 | 5 | 10 | |
| | NeuN | WB | 8 | 8 | 6 | 6 | WB, IHC |
| | | counts | 4 | 6 | 3 | 3 | morphology |
| | Drebin WB | forbrain | 4 | 4 | 3 | 3 | WB, IHC |
| | | | | | | | morphology |
| | Brain wt | 6 mos | 5 | 5 | NA | NA | NA |
| | | 15 mos | 4 | 8 | 7 | 7 | |
| | Open Field | 3 mos | 10 | 21 | 10 | 10 | |
| | | 6 mos | 10 | 15 | 10 | 10 | |
| | | 13 mos | 7 | 10 | 10 | 10 | |
| | Fear Cond | 6 mos | 8 | 11 | NA | NA | |
| | | 11 mos | 16 | 15 | NA | NA | |
| | Spont Alt | 12 mos | 7 | 17 | 10 | 10 | |
| | Barnes Maze | | 14 | 12 | 10 | 9 | |
| | | | | | | CD8 | |
| | | | sham | CD8TCE | TBI | TCE + TBI | |
| C57/BL/6 | Aβ40 (10 wk) | | 8 | 5 | 5 | 9 | NA |
| | P Tau/PHF | | 8 | 5 | 5 | 9 | WB, IHC morph, |
| | | | | | | | huAD IHC |
| | NeuN | | 8 | 5 | 5 | 9 | WB, IHC morph |
| | | | normal | mild AD | severe AD | | |
| Human | microarray | | 9 | 13 | 7 | | GFAP |
| | | | | | | | normalization |
| | Prfl protein | | 5 | 4 | 10 | | WB, huAD IHC |
| | pHLA/APP + | | 10 | NA | 10 | | IHC morph, |
| | anti-CD8 | | | | | | costaining |

Nude mice harboring hiT cells exhibited several similarities with human neurodegenerative disorders, and with Alzheimer's disease in particular. Specifically, early Aβ and later plaque accumulation was evident in them, as was neuroinflammation, silver-stained (fibrillary) neuronal inclusions, synaptic and neuronal loss with brain atrophy, and progressive cognitive impairment. Of these features, several are not found in mice expressing familial Alzheimer's disease gene mutations alone. These most notably include neuronal loss with brain atrophy, and neurofibrillary inclusions. There were, however, distinctions between Alzheimer's disease or FAD-based mouse model neuropathology, and that of nude mice harboring hiT cells: Aβ40 alone was increased without overt involvement of Aβ42, and plaques were predominantly diffuse rather than mature. Nude mice also did not exhibit the acellular “ghost tangles” typically seen in human Alzheimer's disease.

Although patients with the Iowa APP mutation exhibit a pattern of predominant Aβ40 elevation, this feature distinguished hiT cell-bearing nude mice from most transgenic models, as well as from most human Alzheimer's disease. This may be due in part to hiT cell-bearing nude mice exhibiting prominent vascular amyloid, whose chiefly Aβ40 composition may obscure other Aβ species. Mouse Aβ40 also has a reduced efflux rate relative to Aβ42, a pattern opposite to that of human Aβ peptides, and expected to promote greater retention of Aβ40. Factors that specifically inhibit Aβ42 fibrillar assembly in rodent brain may further promote Aβ40 predominance. Ghost tangles, on the other hand, differ from other neurofibrillary structures in that they are comprised mainly of 3R tau, which is virtually absent in adult mice. Hence, a combination of technical and species-specific factors may account for discrepancies between hiT-bearing nude mice and human Alzheimer's disease. Nevertheless, the unique pathology of these mice made further similarities to human disease evident. For example, cognitive impairment in hiT cell-bearing nude mice correlated better with pTau/PHF than with Aβ levels, progressed from hippocampus- to amygdala-dependent tasks, and correlated with brain atrophy in multiple behavioral tests. In this context, it was not surprising that a coupled increase in CD8 and Perforin1 was observed in early-stage Alzheimer's disease brain, which was similar to the increased CD8⁺ T cells with effector activity in hiT-bearing nude mice. Moreover, increased CD8+ T cell reactivity to App_{[471-479]} in severe disease directly paralleled the expansion of those reactive to a nearly identical epitope (App_{[470-478]}) in hiT-bearing nude mice.

Lytic and proinflammatory T cell effector functions affected neuropathological features of hiT cell-bearing mice differently. Perforin1 deficiency prevented CD8+ T cells from remaining in brain, as well as all neuropathological and symptomatic features. In contrast, Ifnγ deficiency allowed the accumulation of amyloid and neurofibrillary structures, albeit with restricted distribution that was reminiscent of an early preclinical stage of Alzheimer's disease. This indicates, as in previous reports, that IFNγ accelerates disease pathology. The impaired astrogliosis and microgliosis in IfnγKO-CD8 group mice is also consistent with earlier work showing that it activates neuroinflammation in distinct Alzheimer's disease models. The unexpected increase in NeuN, Drebrin, and brain mass in IfnγKO-CD8 group mice, however, indicates that IFNγ may also regulate neurodegeneration independent of neuroinflammation. Given these distinct effects, it will be interesting to determine if Perforin1⁺ or IFNγ⁺ CD8+ T cells represent biomarkers for Alzheimer's disease initiation and progression, respectively.

The existence of HLA-DR risk alleles in Parkinson's as well as Alzheimer's disease indicates that T cells may be generally involved in neurodegenerative etiology. Moreover, the recent discovery of lymphatic vasculature in brain demonstrates a potential structural basis for general T cell involvement in brain pathophysiology. Some, but not all previous studies have reported increased CD8+ T cells, or general autoimmune features in Alzheimer's disease, but the nature of T cell involvement has been poorly understood. It is reasonable to speculate that lytic self-reactivity may contribute to hiT cell-induced neuropathology, but this differed from classical autoimmune neurodegeneration characteristic of multiple sclerosis or experimental autoimmune encephalomyelitis in several ways. For example, it was dependent on CD8+ rather than CD4+ T cells, improved rather than impaired by IFNγ deficiency, and did not involve robust immune infiltration. Further, hiT-bearing mice lacked the defining motor deficits of MS and EAE. Taken together with Alzheimer's disease-like neuropathology, this strongly indicates that hiT cell-bearing nude mice do not exhibit MS-like autoimmune neurodegeneration.

It may be possible that hiT cell-mediated neuropathology synergizes with FAD mutations to produce a truly complete Alzheimer's disease-like pathological and symptomatic profile. Addressing whether complete disease-like features in hiT cell-bearing mice can be obtained in strains with human Aβ (App) and/or tau (Mapt) gene knockins, and the general role of strain background in modulating neuropathology, will be similarly important. Most importantly, corroboration of the relevance of hiT cell analogues to sporadic Alzheimer's disease, its prodromal states and risk factors, is critical given their biomarker potential and etiological implications. Regardless, our findings indicate that hiT cells overcome the usual resistance of mice to develop Alzheimer' s disease-like neurodegeneration and neurofibrillary inclusions with aging. This represents the first discrete physiological factor, and the first immunocellular feature of aging, that directly promotes Alzheimer's disease-like neurodegeneration with age.

The findings herein constitute the first evidence that aberrant CD8+ T cells promote tissue degeneration in an age-related pathological condition. It is conceivable that hiT cells reactive to distinct tissue antigens may damage other areas of the brain or body. The hiT model and age-related CD8+ T cell dysfunction in general may thus be relevant to other age-related disorders, and perhaps to the widespread tissue degeneration observed during aging itself.

### Example 2: Standard Operating Procedure for HLA-Peptide Antigen Multimer Staining for Flow Cytometry

Principle

The procedure describes a method used to determine the percentage of CD8+ T cells, T hybridoma cells, or cultured T cells staining positively with MHC I-tumor antigen tetramers. Whole blood or PBMC from human subjects, T hybridoma cells or cultured T cells are aliquoted into U-bottom 96 well microtiter plates, followed by staining with monoclonal antibodies (mAbs). These mAbs, anti-CD8, and anti-KLRG1 or anti-CD103, recognize cell surface markers on a subpopulation of aged T cells. Cells are then stained with pHLA multimers, which recognize antigen-specific receptors on T cells. After incubation, the cells are washed to eliminate the unbound reagents, and they are analyzed using flow cytometry. The percentage of PBMC binding tumor antigen tetramers is determined using the percentage of cells that fall into electronic gates that are defined by control stains.

Specific Requirements

All reagents and equipment are sterilized prior to use; maintenance of sterility throughout procedure is not required, but recommended. Sterile equipment comes in labeled sterile manufacturer packaging, and subsequently is opened and used only in a sterile hood. Equipment can also be sterilized in autoclavable sterilization pouches (Suggested vendor: Fisher, Cat. 91015). All processing must be done in a Laminar Flow Hood approved for human cell Processing. Universal precautions for human tissue handling are required.

Materials/Reagents

Pipets (sterile): P20 eppendorf (VWR pipet, Calibrite INC service) and P200 eppendorf (VWR pipet, Calibrite INC service).

Pipet tips (sterile, filtered): ART 20 μl nuclease/pyrogen free tips (Fisher, 2149P) and ART 200 μl nuclease/pyrogen free tips (Fisher, 2069).

Dulbecco's Phosphate-Buffered Saline (PBS)—sterile (Invitrogen, 14040-133)

FBS—sterile (Gemini Bio. Products, 100-106)

Solution of 2% FBS and 98% PBS—sterile

human Anti-CD8 (PharMingen, #—provided by T-Neuro, Inc.)

human Anti-KLRG1 (PharMingen, #—provided by T-Neuro, Inc.)

human Anti-CD103 (PharMingen, #—provided by T-Neuro, Inc.)

human HLA-tumor antigen tetramers, pentamers, or dextramers (Her-2, Mart-1, gp100)—provided by T-Neuro, Inc.

Coulter, ProImmune, Immudex, various #s

4% Paraformaldehyde (Sigma, 55F-0730)

Solution of 70% Ethanol and 30% Distilled Water

U-bottom 96 microtiter well plates—sterile (Costar, 3595)

FACS tubes—sterile

Container of crushed wet ice with lid

Clean 1 Liter container for bleach

Bleach

Equipment

Refrigerated centrifuge capable of spinning at 1400×g, with at least 2 microtiter plate adapters.

FACScan II or other flow cytometer (minimum 3-color) (Becton Dickinson, Cytomation, or other) Note: Schedule FACS run ahead of time with facility operators if not contracting with CRO.

Quality Control (internal): Record results in the appropriate log books and notebooks. Include observation & signing by witness not performing the procedures if adhering to GLP.

Procedure forms and checklists must be filled out completely (includes equipment log sign-ups) and signed by laboratory supervisor (Quality Assurance supervisor) the same day.

Acquire printout of FACScan flow cytometer settings for the day of the FACS run from the FACS operator. Alternatively, obtain .fcs files for processing & analysis by supervisor.

Procedure

FACS Staining of Cell Surface Markers

1. Using a P200 pipet that has been wiped down with 70% ethanol, transfer 2.5×10⁵ (up to 1×10⁶; equivalent cell # s in all wells) PBMC or whole blood in PBS/2% FBS per well in each of up to 4 wells of a U-bottom, 96 well microtiter plate per tetramer to be analyzed (2 tetramer=4 wells, for example; if only anti-KLRG1 antibody stain is planned, there will be just 2 wells per sample).
Note: The following procedure does not have to be done inside the clinical trial room or laminar flow hood.
2. Using the centrifuge located inside the clinical trial room, D2095, centrifuge the plate at 1400 rpm for 5 minutes.
3. Discard the supernatant by flicking contents into a clean plastic waste container that contains approximately 250-500 ml of bleach. Then blot plate onto a clean paper towel.
4. Transfer the 96 well plate containing cells and antibodies to an ice container filled with wet ice.
5. Using a clean P200 that has been wiped down with 70% ethanol and a sterile pipet tip, resuspend the cells in the wells with 50 μl of the following antibody cocktails:
a) 5 μl anti-CD8^{APC}+5 μl anti-CD8103^{FITC}+35 μl PBS/2% FBS; or substituted with other markers' antibodies;
b) 5 μl anti-CD8^{APC}+5 μl anti-KLRG1^{FITC}+35 μl PBS/2% FBS Include the same 2 cocktails for every HLA-tumor antigen tetramer intended for analysis (2 tetramers+up to 2 stains=up to 4 wells total, for example). Incubate the cells on ice for 30 minutes in the dark (place a lid on the U-bottom microtiter plate and label it so no one knocks it over).
Note: Use a clean pipet tip between each different antibody cocktail administration.
6. Use a clean P200 that has been wiped down with 70% ethanol and a sterile pipet tip to add 200 μl of PBS/2% FBS to each well. Use a clean tip for each well so there is no contamination.
7. Centrifuge the plate for 5 minutes at 1400 rpm with brake on high.
8. Discard the supernatant by flicking contents into a clean plastic waste container that contains approximately 250-500 ml of bleach. Then blot plate onto a clean paper towel.
9. Using a clean P200 that has been wiped down with 70% ethanol and a sterile pipet tip, resuspend the wells previously incubated with each of the three antibody cocktails above with each HLA-tumor antigen tetramer solution (1 well per tetramer to be analyzed; 2 tetramers=2 wells total) as follows:
c) 5 μl HLA-APP-PE+35 μl PBS/2% FBS=1 well
d) 5 μl HLA-empty-PE+35 μl PBS/2% FBS=1 well
10. Incubate the cells at room temp only (25° C.) for 30 minutes in the dark (place a lid on the plate and label it so no one knocks it over).
11. Use a clean P200 that has been wiped down with 70% ethanol and a sterile tip, add 200 μl ice-cold PBS/2% FBS to each well. Use a clean tip for each well so there is no contamination.
12. Using the centrifuge located inside the clinical trial room (D2095), centrifuge the 96 well plate at 1400 rpm for 5 minutes with brake on high.
13. Discard the supernatant by flicking contents into a clean plastic waste container that contains approximately 250-500 ml of bleach. Then blot plate onto a clean paper towel.
14. Using a clean P200 that has been wiped down with 70% ethanol and a sterile pipet tip, resuspend cells in each well with 150 μl ice-cold PBS/2% FBS. Resuspend by trituration. Use a clean tip for each well so there is no contamination.
15. Using a clean P200 that has been wiped down with 70% ethanol and a sterile pipet tip, transfer the cells into FACS tubes for analysis.
16. Optional for delayed analysis of cells (note: only for stained PBMC, not whole blood)
16.1. Using a clean P200 that has been wiped down with 70% ethanol and a sterile pipet tip, fix the cells in the 96 well plate with 50 μl of 4% paraformaldehyde. Make sure that the cells and formaldehyde are mixed well by pipetting up and down inside the tubes, using a different tip for each sample.
17. Take the tubes along with a FACS analysis request form up to the 4th floor, D4029.
18. Obtain the results and store them in the laboratory notebook designated for FACS analysis; download raw .fcs files for further analysis.

Reporting Results

1. All data will be reviewed and approved by the associate responsible for conducting the assay and the laboratory manager no later than 24 hours after FACS results are obtained from the operator.
2. The data analyzed is from a minimum of 50,000 collected events (>250,000 is preferred), and is presented as the proportion of cells within viable lymphocyte gates as determined by forward and side light scatter.
3. Minimum staining must be achieved for both pHLA-empty (>0.7% cells in lymphocyte gate) and anti-CD8 (>3.0% cells in lymphocyte gate) to qualify for further analysis.
4. Procedure forms must be filled out completely (includes check-off for equipment log sign- ups) and signed by laboratory supervisor the same day.

Technical Notes

1. Use only sterile reagents and supplies.
2. Work only in the specified hood. Turn the hoods on 10 minutes prior to use.
3. Wipe down the hood with 70% ethanol prior to use.
4. Don't put a pipet which was used in a flask back into a reagent.
5. Change gloves every time you remove your hands from the hood.
6. Lab coat, gloves, and protective sleeves must be worn when working with any blood components.
7. Cells can be stored at 4 C up to one week prior to analysis by flow cytometry.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A method for treating, inhibiting, reducing the severity of or promoting prophylaxis of age-related cognitive decline, mild cognitive impairment, pathological neurodegeneration, or a combination thereof, in a subject in need thereof, comprising:
administering to the subject a therapeutically effective amount of one more of an inhibitor of cluster of differentiation (CD103), an inhibitor of perforin-1, and an inhibitor of interferon gamma (IFNγ).

2. The method of claim 1, wherein the inhibitor of CD103 is administered, said inhibitor of CD103 is an anti-CD103 antibody and further comprises at least one of: a PE anti-human CD103 antibody from clone Ber-ACTB; a mouse anti-human CD103 monoclonal antibody (mAb) from clone 2G5.1; a humanized antibody of 2G5.1; OX-62; a humanized antibody of OX-62; an anti-mouse CD103 mAb from clone 2E7; a humanized antibody of 2E7; or paxillin.

3. The method of claim 1, wherein the inhibitor of perforin-1 is administered, said inhibitor of perforin-1 comprises a diarylthiophene or GSK2126458.

4. The method of claim 1, wherein the inhibitor of IFNγ is administered, said inhibitor of IFNγ comprises mesopram or rocaglamide.

5. The method of claim 1, wherein the inhibitor of perforin-1 and the inhibitor of IFNγ are administered.

6. The method of claim 1, wherein the inhibitor of CD103, the inhibitor of perforin-1 and the inhibitor of IFNγ are administered.

7. The method of claim 1, wherein the one or more inhibitors modulate CD8+ resident memory T cells (T_{RM}) or effector CD8+ T cells derived therefrom and reduce binding or reaction of said CD8+ T_{RM} or effector CD8+ T cells to an amyloid precursor protein (APP) peptide.

8. The method of claim 7, wherein the reduction in binding or in reaction to an APP peptide comprises reduction in binding or in reaction to an APP peptide of SEQ ID No: 8 of 7 in the brain.

9. The method of claim 1, wherein the subject is a human of an age of at least 50, 55, 60, 65 or 70.

10. The method of claim 7, wherein the effector CD8+ T cells has a reduced activity and/or the CD8+ T_{RM} has a reduced emigration from periphery system into brain, compared to prior to the administration of the one or more inhibitors or compared to a control subject not receiving an administration of the one or more inhibitors.

11. The method of claim 1, wherein the inhibitor of CD103, the inhibitor of perforin-1, and the inhibitor of interferon gamma (IFNγ), independently, comprises an antibody, an antigen-binding fragment of an antibody, a small molecule, or a nucleic acid.

12. The method of claim 1, wherein the pathological neurodegeneration comprises one or more of multiple sclerosis, Parkinson's disease and Alzheimer's disease.

13. The method of claim 1, wherein the age-related cognitive decline or the mild cognitive impairment has one or more symptoms of loss of short-term or long-term memory, decreased ability to maintain focus, and decreased problem solving capacity.

14. The method of claim 1, wherein the subject is a human subject, further comprising identifying the human subject is susceptible to or experiencing a pathological neurodegeneration before the administration, comprising:
detecting an increased presence of CD103+ resident memory CD8+ T cells (CD8+ T_{RM}) in the blood of a human subject, compared to a value obtained from the same human subject of a younger age with no symptoms of age-related cognitive decline, or compared to a value obtained from one or a pool of healthy human subjects with no symptoms of age-related cognitive decline;
wherein the pathological neurodegeneration comprises Parkinson's disease, multiple sclerosis, or Alzheimer's disease; and
wherein the age-related cognitive decline has one or more symptoms of loss of short-term or long-term memory, decreased ability to maintain focus, and decreased problem solving capacity.

15. The method of claim 14, wherein the detecting step further comprises detecting increased levels of CD8A and CD44 in CD103+ CD8+ T_{RM}.

16. The method of claim 14, wherein the human subject is at least 65 years old.

17. A method of treating, inhibiting, reducing the severity or promoting prophylaxis of age-related cognitive decline or of pathological neurodegeneration comprising at least one of multiple sclerosis, Parkinson's disease or Alzheimer's disease in a subject in need thereof, comprising:
administering to the subject a therapeutically effective amount of a vaccine, wherein said vaccine comprises an amyloid precursor protein (APP) peptide selected from the group consisting of SEQ ID Nos: 8, 7, 6, 5, 4, 3, 2 and a combination thereof, or said vaccine comprises APP.

18. The method of claim 17, wherein the subject is a human of an age of at least 40, 50, 60, or 70.

19. A method of identifying a human subject susceptible to or experiencing an age-related cognitive decline or a pathological neurodegeneration, comprising detecting an increased presence of CD103+ resident memory CD8+ T cells (CD8+ T_{RM}) in a blood sample obtained from the human subject with one or more symptoms of loss of short-term or long-term memory, decreased ability to maintain focus, and decreased problem solving capacity.

20. The method of claim 19, wherein the increased presence of CD103+ CD8+ T_{RM} is compared to a value obtained from one or a pool of healthy human subjects with none of the one or more symptoms.
